(19) United States
(12) Patent Application Publication
(10) Pub. No.: US 2025/0079513 A1
(43) Pub. Date: Mar. 6, 2025

(54) SOLID ELECTROLYTES COMPRISING AN IONIC BIFUNCTIONAL MOLECULE, AND USE THEREOF IN ELECTROCHEMISTRY

(71) Applicant: HYDRO-QUÉBEC, Montréal (CA)

(72) Inventors: Benoit FLEUTOT, Boucherville (CA); Xuewei ZHANG, Saint-Hubert (CA); Emmanuelle GARITTE, Montréal (CA)

(21) Appl. No.: 18/726,615

(22) PCT Filed: Jan. 13, 2023
(86) PCT No.: PCT/CA2023/050037

§ 371 Date: Jul. 3, 2024

(30) Foreign Application Priority Data
Jan. 14, 2022 (CA) 3145591

(51) Int. Cl. H01M 10/0565; H01M 4/02; H01M 4/38; H01M 4/58; H01M 4/62; H01M 10/0525; H01M 10/0567; H01M 10/0568

(52) U.S. Cl. CPC H01M 10/0565; H01M 4/382; H01M 4/5825; H01M 4/62; H01M 10/0525; H01M 10/0567; H01M 10/0568; H01M 2004/027; H01M 2004/028

(57) ABSTRACT

The present technology relates to a solid electrolyte comprising inorganic particles and an ionic bifunctional molecule for use in electrochemical applications. Also described are electrochemical cells and batteries comprising said solid electrolyte.

### RELATED APPLICATION

This application claims priority, under applicable law, to Canadian provisional patent application No. 3,145,591 filed on Jan. 14, 2022, the content of which is incorporated herein by reference in its entirety and for all purposes.

### TECHNICAL FIELD

The present application relates to the field of hybrid solid electrolytes comprising a ceramic and to their uses in electrochemical applications. More particularly, the present application relates to ionic compounds, to their manufacturing processes, and to their uses in electrochemical cells, in particular in so-called all-solid-state batteries.

### BACKGROUND

The liquid electrolytes used in lithium-ion batteries are flammable and slowly degrade to form a passivation layer on the surface of the lithium film or solid electrolyte interface (SEI), irreversibly consuming lithium, which reduces the coulombic efficiency of the battery. In addition, lithium anodes undergo significant morphological changes during battery cycling and lithium dendrites are formed. As these usually migrate through the electrolyte, they can eventually cause short circuits.

Safety concerns and the requirement for higher energy density have spurred research into the development of an all-solid-state rechargeable lithium battery with a polymer, ceramic or polymer-ceramic hybrid electrolyte, all three of which are more stable towards metallic lithium and reduce the growth of lithium dendrites.

However, the field of application of solid electrolytes is still limited. Indeed, solid electrolytes present problems related to their limited electrochemical stability, their limited interfacial stability, their relatively low ionic conductivity, loss of reactivity, poor contact between solid interfaces, etc.

Consequently, there is a need for the development of all-solid-state electrochemical systems that exclude one or more of the disadvantages of conventional all-solid-state electrochemical systems.

### SUMMARY

According to a first aspect, the present technology relates to a solid electrolyte comprising inorganic particles and an ionic bifunctional molecule of Formulae I or II:

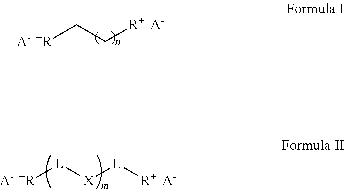

wherein,
A⁻ is a delocalized anion;
R⁺ is selected from —N⁺(R₁R₂R₃) and —P⁺(R₁R₂R₃) groups;
R₁, R₂, and R₃ are independently selected from a substituted or unsubstituted linear or branched C₁₋₁₂alkyl group; or R₁ and R₂ together with the nitrogen or phosphorus atom form a heterocycle having one or more rings and having from 3 to 12 members and R₃ is as previously defined; or R₁, R₂, and R₃ together with the nitrogen or phosphorus atom form a partially unsaturated heterocycle or heteroaromatic having one or more rings and having from 5 to 12 members;
L is a linear or branched C₂₋₄alkylene;
X is O or S;
m is a number in the range from 1 to 6; and
n is a number in the range from 1 to 11.

More particularly, the present technology relates to a solid electrolyte comprising inorganic particles and an ionic bifunctional molecule of Formulae I or II:

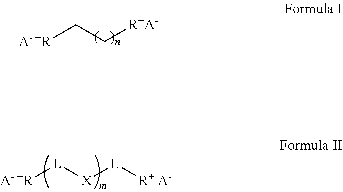

wherein,
A⁻ is a delocalized anion;
R⁺ is selected from —N⁺(R₁R₂R₃) and —P⁺(R₁R₂R₃) groups excluding cations derived from amidine, guanidine and phosphazene superbases;
R₁, R₂, and R₃ are independently selected from a substituted or unsubstituted linear or branched C₁₋₁₂alkyl group; or R₁ and R₂ together with the nitrogen or phosphorus atom form a heterocycle having one or more rings and having from 3 to 12 members and R₃ is as previously defined; or R₁, R₂, and R₃ together with the nitrogen or phosphorus atom form a partially unsaturated heterocycle or heteroaromatic having one or more rings and having from 5 to 12 members;
L is a linear or branched C₂₋₄alkylene;
X is O or S;
m is a number in the range from 1 to 6; and
n is a number in the range from 1 to 11.

According to an embodiment, the delocalized anion is selected from hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), 2-trifluoromethyl-4,5-dicyanoimidazolate (TDI⁻), 4,5-dicyano-1,2,3-triazolate (DCTA⁻), bis(pentafluoroethylsulfonyl)imide (BETI⁻), difluorophosphate (DFP⁻), tetrafluoroborate (BF₄⁻), bis(oxalato)borate (BOB⁻), nitrate (NO₃⁻), perchlorate (ClO₄⁻), hexafluoroarsenate (AsF₆⁻), trifluoromethanesulfonate (CF₃SO₃⁻ or ⁻OTf), fluoroalkylphosphate ([PF₃(CF₂CF₃)₃]⁻ or FAP⁻), tetrakis(trifluoroacetoxy)borate ([B(OCOCF₃)₄]⁻ or TFAB⁻), bis(1,2-benzenediolato(2-)-O,O′)borate ([B(C₆O₂)₂]⁻ or BBB⁻), difluoro(oxalato)borate (BF₂(C₆O₄)⁻ or FOB⁻), and an anion of formula C₆O₄R^{x} (R^{x}═C₂₋₄alkyl).

According to an example, the delocalized anion is selected from hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), tetrafluoroborate (BF₄⁻), and trifluoromethanesulfonate (CF₃SO₃⁻ or ⁻OTf).

According to an example of interest, the delocalized anion is bis(trifluoromethanesulfonyl)imide (TFSI⁻).

According to some embodiments, R⁺ is an —N⁺(R₁R₂R₃) group.

According to an example, R₁, R₂, and R₃ are independently selected from substituted or unsubstituted linear or branched C₁₋₁₂alkyl groups.

According to another example, R₁, R₂, and R₃ are independently selected from linear or branched C₁₋₁₂alkyl groups, or at least one of R₁, R₂, or R₃ is substituted by a halogen atom or an alkoxyl, ether, ester, or siloxy group.

According to another example, R₁ and R₂ together with the nitrogen atom form a heterocycle having one or more rings and having from 3 to 12 members and R₃ is as previously defined, preferably R₃ is a C₁₋₁₂ alkyl or a C₁₋₄alkyl.

According to another example, R₁, R₂, and R₃ together with the nitrogen atom form a partially unsaturated heterocycle or heteroaromatic having one or more rings and having 5 to 12 members.

According to another example, R⁺ is selected from:

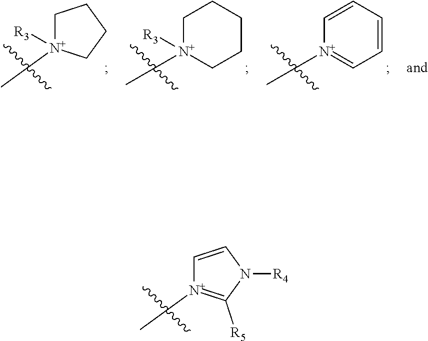

wherein R₃ is as previously defined, R₄ is a substituted or unsubstituted linear or branched C₁₋₁₂alkyl, C₁₋₁₂alkenyl, or C₁₋₁₂alkynyl group, R₅ is a hydrogen atom, or a substituted or unsubstituted linear or branched C₁₋₁₂alkyl, C₁₋₁₂alkenyl, or C₁₋₁₂ alkynyl group, and the heterocycle is optionally substituted.

According to an example, R₄ is a C₁₋₄alkyl group.

According to another example, R₅ is a C₁₋₄alkyl group.

According to another example, R₃ is an unsubstituted C₁₋₄alkyl group. According to an example of interest, R₃ is selected from a methyl group, an ethyl group, an n- or i-propyl group, and an n-, i-, s-, or t-butyl group.

According to some other embodiments, R⁺ is a —P⁺(R₁R₂R₃) group.

According to an example, R₁, R₂, and R₃ are independently selected from substituted or unsubstituted linear or branched C₁₋₁₂alkyl groups.

According to another example, R₁, R₂, and R₃ are independently selected from linear or branched C₁₋₁₂alkyl groups, or at least one of R₁, R₂, or R₃ is substituted by a halogen atom or an alkoxyl, ether, ester, or siloxy group.

According to another embodiment, n is a number in the range from 2 to 10, or from 3 to 8, or from 4 to 6.

According to another embodiment, the ionic bifunctional molecule is 1,1′-(1,6-hexamethylene) bis(1-methylpyrrolidinium) bis(trifluoromethanesulfonyl)imide.

According to another embodiment, the ionic bifunctional molecule is 1,1′-(1,12-dodecamethylene) bis(1-methylpyrrolidinium) bis(trifluoromethanesulfonyl)imide.

According to another embodiment, the ionic bifunctional molecule is 1,1′-(2,2′-(ethylenedioxy)diethane) bis(1-methylpyrrolidinium) bis(trifluoromethanesulfonyl)imide.

According to another embodiment, the ionic bifunctional molecule is 1,1′-(thiol bis(1,2-ethane)) bis(1-methylpyrrolidinum) bis(trifluoromethanesulfonyl)imide.

According to another embodiment, the ionic bifunctional molecule is 3,3′-(1,6-hexamethylene) bis(1,2-dimethylimidazolium) bis(trifluoromethanesulfonyl)imide.

According to another embodiment, the ionic bifunctional molecule is at a concentration of from about 0.5 wt. % to about 50 wt. %, or from about 2 wt. % to about 30 wt. %, or from about 4 wt. % to about 20 wt. %, or from about 5 wt. % to about 15 wt. % in the solid electrolyte.

According to another embodiment, the inorganic particles comprise a material selected from glasses, glass-ceramics, ceramics, nanoceramics, and a combination of at least two thereof.

According to some preferred embodiments, the inorganic particles comprise a fluoride-, phosphide-, sulfide-, oxysulfide- or oxide-based ceramic, glass, or glass-ceramic.

According to some preferred embodiments, the inorganic particles comprise a LISICON, thio-LISICON, argyrodite, garnet, NASICON, perovskite, oxide, sulfide, oxysulfide, phosphide, or fluoride compound in crystalline and/or amorphous form, or a combination of at least two thereof.

According to some preferred embodiments, the inorganic particles comprise a compound selected from inorganic compounds of formulae MLZO (for example, M₇La₃Zr₂O₁₂, M₍₇₋ₐ₎La₃Zr₂Al_{b}O₁₂, M₍₇₋ₐ₎La₃Zr₂Ga_{b}O₁₂, M₍₇₋ₐ₎La₃Zr_{(2-b)}Ta_{b}O₁₂, and M₍₇₋ₐ₎La₃Zr_{(2-b)}Nb_{b}O₁₂); MLTaO (for example, M₇La₃Ta₂O₁₂, M₅La₃Ta₂O₁₂, and M₆La₃Ta_{1.5}Y_{0.5}O₁₂); MLSnO (for example, M₇La₃Sn₂O₁₂); MAGP (for example, M₁₊ₐAlₐGe₂₋ₐ(PO₄)₃); MATP (for example, M₁₊ₐAlₐTi₂₋ₐ(PO₄)₃); MLTiO (for example, M₃ₐLa_{(2/3-a)}TiO₃); MZP (for example, MₐZr_{b}(PO₄)_{c}); MCZP (for example, MₐCa_{b}Zr_{c}(PO₄)_{d}); MGPS (for example, MₐGe_{b}P_{c}S_{d} such as M₁₀GeP₂S₁₂); MGPSO (for example, MₐGe_{b}P_{c}S_{d}Oₑ); MSiPS (for example, MₐSi_{b}P_{c}S_{d} such as M₁₀SiP₂S₁₂); MSiPSO (for example, MₐSi_{b}P_{c}S_{d}Oₑ); MSnPS (for example, MₐSn_{b}P_{c}S_{d} such as M₁₀SnP₂S₁₂); MSnPSO (for example, MₐSn_{b}P_{c}S_{d}Oₑ); MPS (for example, MₐP_{b}S_{c} such as M₇P₃S₁₁); MPSO (for example, MₐP_{b}S_{c}O_{d}); MZPS (for example, MₐZn_{b}P_{c}S_{d}); MZPSO (for example, MₐZn_{b}P_{c}S_{d}Oₑ); xM₂S-yP₂S₅; xM₂S-yP₂S₅-zMX; xM₂S-yP₂S₅-zP₂O₅; xM₂S-yP₂S₅-zP₂O₅-wMX; xM₂S-yM₂O-zP₂S₅; xM₂S-yM₂O-zP₂S₅-wMX; xM₂S-yM₂O-zP₂S₅-wP₂O₅; xM₂S-yM₂O-zP₂S₅-wP₂O₅-vMX; xM₂S-ySiS₂; MPSX (for example, MₐP_{b}S_{c}X_{d} such as M₇P₃S₁₁X, M₇P₂S₈X, and M₆PS₅X); MPSOX (for example, MₐP_{b}S_{c}O_{d}Xₑ); MGPSX (MₐGe_{b}P_{c}S_{d}Xₑ); MGPSOX (MₐGe_{b}P_{c}S_{d}OₑX_{f}); MSiPSX (MₐSi_{b}P_{c}S_{d}Xₑ); MSiPSOX (MₐSi_{b}P_{c}S_{d}OₑX_{f}); MSnPSX (MₐSn_{b}P_{c}S_{d}Xₑ); MSnPSOX (MₐSn_{b}P_{c}S_{d}OₑX_{f}); MZPSX (MₐZn_{b}P_{c}S_{d}Xₑ); MZPSOX (MₐZn_{b}P_{c}S_{d}OₑX_{f}); M₃OX; M₂HOX; M₃PO₄; M₃PS₄; and MₐPO_{b}N_{c} (where a=2b+3c−5);
wherein,
M is an alkali metal ion, an alkaline earth metal ion, or a combination of at least two thereof, and wherein when M comprises an alkaline earth metal ion, then the number of M is adjusted to achieve electroneutrality;
X is selected from F, Cl, Br, I, or a combination of at least two thereof;
a, b, c, d, e, and f are numbers other than zero and are, independently in each formula, selected to achieve electroneutrality; and
v, w, x, y, and z are numbers other than zero and are, independently in each formula, selected to obtain a stable compound.

According to an example, M is selected from Li, Na, K, Rb, Cs, Be, Mg, Ca, Sr, Ba, or a combination of at least two thereof. For example, M is Li.

According to another example, the inorganic particles comprise an inorganic compound of formula MATP.

According to another example, the inorganic particles comprise an argyrodite-type inorganic compound of formula Li₆PS₅X, wherein X is Cl, Br, I, or a combination of at least two thereof.

According to another example, the inorganic particles comprise an inorganic compound of formula Li₆PS₅Cl.

According to another embodiment, the inorganic particles are present at a concentration of from about 25 wt. % to about 95 wt. %, or from about 40 wt. % to about 90 wt. %, or from about 60 wt. % to about 90 wt. % in the solid electrolyte.

According to another embodiment, the ratio “inorganic particles:ionic bifunctional molecule” by weight is in the range from 2:1 to 30:1, or from 3:1 to 20:1, or from 5:1 to 15:1.

According to another embodiment, the solid electrolyte also comprises a polymer.

According to an example, the polymer is a linear or branched polymer selected from polyethers, polythioethers, polyesters, polythioesters, poly(dimethylsiloxanes), poly(alkylene carbonate), poly(alkylene thiocarbonate), poly(alkylene sulfones), poly(alkylene sulfamides), polyimides, polyamides, polyphosphazenes, polyurethanes, poly(vinyl alcohols), polyacrylonitriles, polyethacrylates, polymethacrylates, and copolymers thereof.

According to an example, the polyether is poly(ethylene oxide) (PEO), poly(propylene oxide) (POP), or a copolymer (EO/PO).

According to another example, the crosslinkable functional group is selected from acrylate, methacrylate, vinyl, glycidyl, and mercapto functional groups.

According to another example, the polymer is the reaction product of at least one monomer comprising at least one polymerizable or crosslinkable function and a compound comprising at least one SH functional group.

According to another embodiment, the polymer is present at a concentration of from about 0.1 wt. % to about 20 wt. %, or from about 1 wt. % to about 15 wt. %, or from about 2 wt. % to about 10 wt. % in the solid electrolyte.

According to another embodiment, the solid electrolyte also comprises an additive.

According to an example, the additive is a fluorinated compound comprising an amide function. For example, the fluorinated compound is of formula R⁶X⁶C(O)N(H)X⁷R⁷, where R⁶ and R⁷ are independently alkyl, cycloalkyl, heterocycloalkyl, aryl, or heteroaryl groups, X⁶ is O, NH, or is absent, and X⁷ is absent or is a C(O), S(O)₂, or Si(R⁸R⁹) group, where R⁸ and R⁹ are alkyl groups, and where at least one of R⁶, R⁷, R⁸, and R⁹ is a group substituted by one or more fluorine atoms. According to an example of interest, R⁶ is a perfluorinated group and X⁶ is absent.

According to another example, the additive is present at a concentration of from about 5 wt. % to about 40 wt. %, or from about 10 wt. % to about 35 wt. %, or from about 15 wt. % to about 30 wt. % in the solid electrolyte.

According to another aspect, the present technology relates to an electrochemical cell comprising a negative electrode, a positive electrode, and an electrolyte, wherein the electrolyte is as herein defined.

According to an embodiment, the positive electrode comprises a positive electrode material comprising a positive electrode electrochemically active material.

According to an example, the positive electrode material is on a current collector.

According to another example, the positive electrode electrochemically active material is selected from metal phosphates, lithiated metal phosphates, metal oxides, and lithiated metal oxides.

According to another example, the positive electrode electrochemically active material is LiM′PO₄ where M′ is Fe, Ni, Mn, Co, or a combination of at least two thereof, LiV₃O₈, V₂O₅F, LiV₂O₅, LiMn₂O₄, LiM″O₂, where M″ is Mn, Co, Ni, or a combination of at least two thereof (such as NMC, LiMnₓCo_{y}Ni_{z}O₂ with x+y+z=1), Li(NiM′″)O₂ (where M″′ is Mn, Co, Al, Fe, Cr, Ti, Zr, or a combination of at least two thereof), sulfur, elemental selenium, elemental iodine, iron(III) fluoride, copper(II) fluoride, lithium iodide, carbon-based active materials such as graphite, organic cathode active materials, or a combination of at least two thereof, when compatible with each other.

According to another embodiment, the positive electrode material further comprises an electronically conductive material, a binder, a salt, an ionic bifunctional molecule, and/or inorganic particles.

According to another embodiment, the negative electrode comprises a negative electrode material comprising a negative electrode electrochemically active material.

According to an example, the negative electrode material is on a current collector.

According to some preferred embodiments, the negative electrode electrochemically active material comprises a metal film comprising an alkali or alkaline earth metal or an alloy comprising an alkali or alkaline earth metal. According to an example, the alkali metal is selected from lithium and sodium.

According to further preferred embodiments, the negative electrode electrochemically active material comprises an intermetallic compound (for example, SnSb, TiSnSb, Cu₂Sb, AlSb, FeSb₂, FeSn₂, and CoSn₂), metal oxide, metal nitride, metal phosphide, metal phosphate (for example, LiTi₂(PO₄)₃), metal halide (for example, metal fluoride), metal sulfide, metal oxysulfide, carbon (for example, graphite, graphene, reduced graphene oxide, hard carbon, soft carbon, exfoliated graphite, and amorphous carbon), silicon (Si), a silicon-carbon composite (Si—C), a silicon oxide (SiOₓ), a silicon oxide-carbon composite (SiOₓ—C), tin (Sn), a tin-carbon composite (Sn—C), a tin oxide (SnOₓ), a tin oxide-carbon composite (SnOₓ—C), and combinations thereof, when compatible. According to an example, the metal oxide is selected from compounds of formulae M″″_{b}O_{c} (where M″″ is Ti, Mo, Mn, Ni, Co, Cu, V, Fe, Zn, Nb, or a combination thereof; and b and c are numbers such that the ratio c:b is in the range from 2 to 3) (for example, MoO₃, MoO₂, MoS₂, V₂O₅, and TiNb₂O₇), spinel oxides (for example, NiCo₂O₄, ZnCo₂O₄, MnCo₂O₄, CuCo₂O₄, and CoFe₂O₄) and LiM′″″O (where M′″″ is Ti, Mo, Mn, Ni, Co, Cu, V, Fe, Zn, Nb, or a combination thereof) (for example, lithium titanate (such as Li₄Ti₅O₁₂) or lithium molybdenum oxide (such as Li₂Mo₄O₁₃)). According to an example of interest, the negative electrode material further comprises an electronically conductive material, a binder, a salt, an ionic bifunctional molecule, and/or inorganic particles.

According to another aspect, the present technology relates to a battery comprising at least one electrochemical cell as herein defined.

According to an embodiment, said battery is selected from the group consisting of a lithium battery, a lithium-ion battery, a sodium battery, a sodium-ion battery, a potassium battery, a potassium-ion battery, a magnesium battery, and a magnesium-ion battery. According to an example of interest, said battery is a lithium battery. According to another example of interest, said battery is a lithium-ion battery.

### BRIEF DESCRIPTION OF THE FIGURES

FIG. 1 presents the results of differential scanning calorimetry analysis obtained for Salts 1, 2, 4 and 5, as described in Example 3.

FIG. 2 presents the results of the thermogravimetric analysis obtained for Salts 1 to 5, as described in Example 3.

FIG. 3 is a graph presenting linear sweep voltammetry curves obtained for cells comprising the electrolytes E1 to E3, as described in Example 4(b).

FIG. 4 is a graph presenting cyclic voltammetry curves obtained for cells comprising electrolytes E2 and E3, as described in Example 4(b).

FIG. 5 presents images of a lithium foil respectively dipped in (A) tetraethylene glycol dimethyl ether (TEGDME), (B) a solution of 1-Butyl-1-methylpyrrolidinium bis(trifluoromethylsulfonyl)imide ([PYR1,4]TFSI) in TEGDME, and (C) a solution of Salt 1 in TEGDME, as described in Example 4(c).

FIG. 6 presents images of a solid electrolyte pellet, as described in Example 5(a).

FIG. 7 is a graph presenting ionic conductivity results as a function of temperature for Cells 4 ( [symbol] ), 5 (▴), 6 (●), and 7 (★), as described in Example 6(b).

FIG. 8 is a graph presenting ionic conductivity results as a function of temperature for Cells 8 ( [symbol] ), 9 (●), 10 (▴), and 11 (▾), as described in Example 6(b).

FIG. 9 presents scanning electron microscopy (SEM) images of the ceramic-ionic plastic salt composite solid electrolyte film E6 in (A) before creep, and in (B) and (C) after creep at a temperature of 70° C., as described in Example 6(c).

FIG. 10 is a graph presenting ionic conductivity results as a function of temperature for Cells 12 (●) and 13 (▾), as described in Example 7(b).

### DETAILED DESCRIPTION

All technical and scientific terms and expressions used herein have the same definitions as those generally understood by the person skilled in the art of the present technology. The definition of some terms and expressions used is nevertheless provided below.

When the term “about” is used herein, it means approximately, in the region of, or around. For example, when the term “about” is used in relation to a numerical value, it modifies it by a variation of 10% above and below its nominal value. This term can also take into account, for example, the experimental error of a measuring device or rounding.

When a range of values is mentioned in the present application, the lower and upper limits of the range are, unless otherwise indicated, always included in the definition. When a range of values is mentioned in the present application, then all intermediate ranges and subranges, as well as the individual values included in the ranges of values, are included in the definition.

When the article “a” is used to introduce an element in the present application, it does not have the meaning of “only one”, but rather of “one or more”. Of course, where the description states that a particular step, component, element, or feature “may” or “could” be included, that particular step, component, element, or feature is not required to be included in each embodiment.

The chemical structures described herein are drawn according to the conventions of the field. Also, when an atom, such as a carbon atom, as drawn seems to include an incomplete valence, then it is assumed that the valence is satisfied by one or more hydrogen atoms even if they are not explicitly drawn.

The term “alkyl” as used herein refers to saturated hydrocarbons having from 1 to 12 carbon atoms, including linear or branched alkyl groups. Non-limiting examples of alkyl groups can include methyl, ethyl, propyl, butyl, pentyl, hexyl, heptyl, octyl, nonyl, decyl, isopropyl, tert-butyl, sec-butyl, isobutyl, and so on. When the alkyl group is located between two functional groups, then the term alkyl also includes alkylene groups such as methylene, ethylene, propylene, and so on. The terms “Cₘ-Cₙalkyl” and “Cₘ-Cₙalkylene” respectively refer to an alkyl or alkylene group having from the indicated number “m” to the indicated number “n” of carbon atoms.

The term “cycloalkyl” as used herein refers to a group comprising one or more saturated or partially unsaturated (non-aromatic) carbocyclic rings comprising from 3 to 15 members in a monocyclic or polycyclic system, including spiro (sharing an atom), fused (sharing at least one bond), or bridged carbocycles and can be optionally substituted. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, and so on. When the cycloalkyl group is located between two functional groups, the term cycloalkylene can also be used.

The term “heterocycloalkyl” as used herein refers to a group comprising a saturated or partially unsaturated (non-aromatic) carbocyclic ring comprising from 3 to 15 members in a monocyclic or polycyclic system, including spiro (sharing an atom), fused (sharing at least one bond), or bridged and can be optionally substituted, and having, carbon atoms and from 1 to 4 heteroatoms (for example, N, O, S, or P) or groups containing such heteroatoms (for example, NH, NRₓ (Rₓ is an alkyl, acyl, aryl, heteroaryl, or cycloalkyl group), PO₂, SO, SO₂, and other similar groups). Heterocycloalkyl groups can be bonded to a carbon atom or a heteroatom (for example via a nitrogen atom) where possible. The term heterocycloalkyl includes both unsubstituted heterocycloalkyl groups and substituted heterocycloalkyl groups. When the heterocycloalkyl group is located between two functional groups, the term heterocycloalkylene can also be used.

The terms “aryl” or “aromatic” refer to an aromatic group having 4n+2 conjugated π(pi) electrons in which n is a number from 1 to 3, in a monocyclic group, or a fused bicyclic or tricyclic system having a total of from 6 to 15 ring members, in which at least one of the rings of a system is aromatic. The terms “aryl” or “aromatic” refer to both conjugated monocyclic and polycyclic systems. The terms “aryl” or “aromatic” also include substituted or unsubstituted groups. Examples of aryl groups include, without limitation, phenyl, benzyl, phenethyl, 1-phenylethyl, tolyl, naphthyl, biphenyl, terphenyl, indenyl, benzocyclooctenyl, benzocycloheptenyl, azulenyl, acenaphthylenyl, fluorenyl, phenanthrenyl, anthracenyl, perylenyl, and so on.

The terms “heteroaryl”, “heteroarylene”, or “heteroaromatic” refer to an aromatic group having 4n+2 conjugated π(pi) electrons in which n is a number from 1 to 3, for example having from 5 to 18 ring atoms, preferably 5, 6, or 9 ring atoms in a conjugated monocyclic or polycyclic system (fused or not); and having, in addition to carbon atoms, from 1 to 6 heteroatoms selected from oxygen, nitrogen and sulfur or groups containing such heteroatoms (for example, NH and NRₓ (Rₓ is alkyl, acyl, aryl, heteroaryl, or cycloalkyl group), SO, and other similar groups). A polycyclic ring system comprises at least one heteroaromatic ring. Heteroaryls can be directly attached, or linked via a C₁-C₃alkyl group (also called heteroarylalkyl or heteroaralkyl). Heteroaryl groups can be linked to a carbon atom or a heteroatom (for example, via a nitrogen atom), where possible.

In general, the term “substituted” means that one or more hydrogen atom(s) on the designated group is replaced by a suitable substituent. The substituents or combinations of substituents contemplated in the present description are those resulting in the formation of a chemically stable compound. Examples of substituents include halogen atoms (such as fluorine) and hydroxyl, oxo, alkyl, alkoxyl, alkoxyalkyl, nitrile, azido, carboxylate, alkoxycarbonyl, alkylcarbonyl, primary, secondary, or tertiary amine, amide, nitro, silane, siloxane, thiocarboxylate, sulfonyl, sulfonate, alkenyl, alkynyl, aryl, heteroaryl, cycloalkyl, heterocycloalkyl groups, or a combination thereof.

The present technology generally relates to a solid electrolyte and its use in electrochemical applications. For example, the solid electrolyte can be a primarily inorganic solid electrolyte or a polymer-ceramic hybrid solid electrolyte.

The present technology relates more particularly to a solid electrolyte comprising inorganic particles and an ionic bifunctional molecule of Formulae I or II:

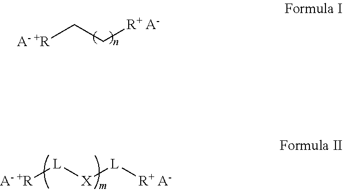

wherein,
A⁻ is a delocalized anion;
R⁺ is selected from —N⁺(R₁R₂R₃) and —P⁺(R₁R₂R₃) groups, excluding cations derived from amidine, guanidine and phosphazene superbases;
R₁, R₂, and R₃ are independently selected from a substituted or unsubstituted linear or branched C₁₋₁₂alkyl group; or R₁ and R₂ together with the nitrogen or phosphorus atom form a heterocycle having one or more rings and having from 3 to 12 members and R₃ is as previously defined; or R₁, R₂, and R₃ together with the nitrogen or phosphorus atom form a partially unsaturated heterocycle or heteroaromatic having one or more rings and having from 5 to 12 members;
L is a linear or branched C₂₋₄alkylene;
X is O or S;
m is a number in the range from 1 to 6; and
n is a number in the range from 1 to 11.

The delocalized anion can be selected from the group consisting of hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), 2-trifluoromethyl-4,5-dicyanoimidazolate (TDI⁻), 4,5-dicyano-1,2,3-triazolate (DCTA⁻), bis(pentafluoroethylsulfonyl)imide (BETI⁻), difluorophosphate (DFP⁻), tetrafluoroborate (BF₄⁻), bis(oxalato)borate (BOB⁻), nitrate (NO₃⁻), perchlorate (ClO₄⁻), hexafluoroarsenate (AsF₆⁻), trifluoromethanesulfonate (CF₃SO₃⁻ or ⁻OTf), fluoroalkylphosphate ([PF₃(CF₂CF₃)₃]⁻ or FAP⁻), tetrakis(trifluoroacetoxy)borate ([B(OCOCF₃)₄]⁻ or TFAB⁻), bis(1,2-benzenediolato(2-)-O,O′)borate ([B(C₆O₂)₂]⁻ or BBB⁻), difluoro(oxalato)borate (BF₂(C₆O₄)⁻ or FOB⁻), and an anion of formula C₆O₄R^{x} (R^{x}═C₂₋₄alkyl). For example, the delocalized anion is selected from the group consisting of hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), tetrafluoroborate (BF₄⁻), and trifluoromethanesulfonate (CF₃SO₃⁻ or ⁻OTf).

According to an example, R⁺ is a group of formula —N⁺(R₁R₂R₃), wherein R₁, R₂, and R₃ are independently selected from substituted or unsubstituted linear or branched C₁₋₁₂alkyl groups.

According to another example, R⁺ is a group of formula —N⁺(R₁R₂R₃), wherein R₁, R₂, and R₃ are independently selected from linear or branched C₁₋₁₂alkyl groups, or at least one of R₁, R₂, or R₃ is substituted by a halogen atom or an alkoxyl, ether, ester, or siloxy group.

According to another example, R⁺ is a group of formula —N⁺(R₁R₂R₃), wherein R₁ and R₂ together with the nitrogen atom form a heterocycle having one or more rings and having from 3 to 12 members and R₃ is as previously defined, preferably R₃ is a C₁₋₁₂alkyl group or a C₁₋₄alkyl group. According to an example of interest, R₃ is an unsubstituted C₁₋₄alkyl group (such as methyl, ethyl, n- or i-propyl, n-, i-, s-, and t-butyl), and preferably R₃ is a methyl group.

According to another example, R⁺ is a group of formula —N⁺(R₁R₂R₃), wherein R₁, R₂, and R₃ together with the nitrogen atom form a partially unsaturated heterocycle or heteroaromatic having one or more rings and having from 5 to 12 members.

According to another example, R⁺ is selected from:

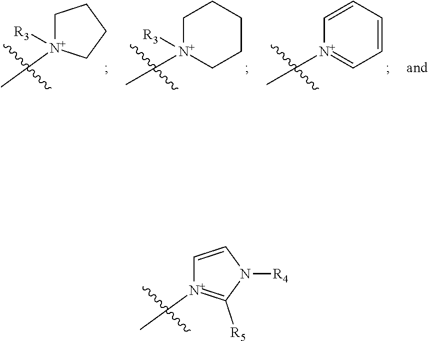

wherein,
R₃ is as previously defined, R₄ is selected from a substituted or unsubstituted linear or branched C₁₋₁₂alkyl, C₁₋₁₂alkenyl, and C₁₋₁₂alkynyl group, preferably C₁₋₄alkyl, R₅ is a hydrogen atom or a C₁₋₁₂alkyl group, substituted or unsubstituted linear or branched C₁₋₁₂alkenyl or C₁₋₁₂alkynyl, preferably C₁₋₄alkyl, preferably R₃ is an unsubstituted C₁₋₄alkyl group (such as methyl, ethyl, n- or i-propyl, n-, i-, s-, and t-butyl, preferably methyl); and
the heterocycle is optionally substituted.

According to another example, R⁺ is a group of formula —P⁺(R₁R₂R₃), wherein R₁, R₂, and R₃ are independently selected from substituted or unsubstituted linear or branched C₁₋₁₂alkyl groups.

According to another example, R⁺ is a group of formula —P⁺(R₁R₂R₃), wherein R₁, R₂, and R₃ are independently selected from linear or branched C₁₋₁₂alkyl groups, or at least one of R₁, R₂, or R₃ is substituted by a halogen atom or an alkoxyl, ether, ester, or siloxy group.

According to some examples, n can be a number in the range from 2 to 10, or from 3 to 8, or from 4 to 6, upper and lower limits included.

According to some examples, the ionic bifunctional molecule is selected from 1,1′-(1,6-hexamethylene) bis(1-methylpyrrolidinium) bis(trifluoromethanesulfonyl)imide, 1,1′-(1,12-dodecamethylene) bis(1-methylpyrrolidinium) bis(trifluoromethanesulfonyl)imide, 1,1′-(2,2′-(ethylenedioxy)diethane) bis(1-methylpyrrolidinium) bis(trifluoromethanesulfonyl)imide, 1,1′-(thiol bis(1,2-ethane)) bis(1-methylpyrrolidinum), and 3,3′-(1,6-hexamethylene) bis(1,2-dimethylimidazolium) bis(trifluoromethanesulfonyl)imide. According to an example of interest, the ionic bifunctional molecule is 1,1′-(1,6-hexamethylene) bis(1-methylpyrrolidinium) bis(trifluoromethanesulfonyl)imide.

The ionic bifunctional molecule can be present in the electrolyte at a concentration in the range from about 0.5 wt. % to about 50 wt. %, upper and lower limits included. For example, the ionic bifunctional molecule can be present in the electrolyte at a concentration in the range from about 2 wt. % to about 30 wt. %, or from about 4 wt. % to about 20 wt. %, or from about 5 wt. % to about 15 wt. %, upper and lower limits included.

The inorganic particles can be selected from any known inorganic solid electrolyte material particles and can be selected according to their compatibility with the various elements of an electrochemical cell. For example, the inorganic particles can comprise a material selected from glasses, glass-ceramics, ceramics, nanoceramics, and a combination of at least two thereof.

According to an example, the inorganic particles can comprise a fluoride-, phosphide-, sulfide-, oxysulfide- or oxide-based ceramic, glass, or glass-ceramic.

According to another example, the inorganic particles can comprise a LISICON, thio-LISICON, argyrodite, garnet, NASICON, perovskite, oxide, sulfide, oxysulfide, phosphide or fluoride compound in crystalline and/or amorphous form, or a combination of at least two thereof.

According to another example, the inorganic particles comprise a compound selected from the inorganic compounds of formulae:
MLZO (for example, M₇La₃Zr₂O₁₂, M₍₇₋ₐ₎La₃Zr₂Al_{b}O₁₂, M₍₇₋ₐ₎La₃Zr₂Ga_{b}O₁₂, M₍₇₋ₐ₎La₃Zr_{(2-b)}Ta_{b}O₁₂, and M₍₇₋ₐ₎La₃Zr_{(2-b)}Nb_{b}O₁₂);
MLTaO (for example, M₇La₃Ta₂O₁₂, M₅La₃Ta₂O₁₂, and M₆La₃Ta_{1.5}Y_{0.5}O₁₂);
MLSnO (for example, M₇La₃Sn₂O₁₂);
MAGP (for example, M₁₊ₐAlₐGe₂₋ₐ(PO₄)₃);
MATP (for example, M₁₊ₐAlₐTi₂₋ₐ(PO₄)₃);
MLTiO (for example, M₃ₐLa_{(2/3-a)}TiO₃);
MZP (for example, MₐZr_{b}(PO₄)_{c});
MCZP (for example, MₐCa_{b}Zr_{c}(PO₄)_{d});
MGPS (for example, MₐGe_{b}P_{c}S_{d} such as M₁₀GeP₂S₁₂);
MGPSO (for example, MₐGe_{b}P_{c}S_{d}Oₑ);
MSiPS (for example, MₐSi_{b}P_{c}S_{d} such as M₁₀SiP₂S₁₂);
MSiPSO (for example, MₐSi_{b}P_{c}S_{d}Oₑ);
MSnPS (for example, MₐSn_{b}P_{c}S_{d} such as M₁₀SnP₂S₁₂);
MSnPSO (for example, MₐSn_{b}P_{c}S_{d}Oₑ);
MPS (for example, MₐP_{b}S_{c} such as M₇P₃S₁₁);
MPSO (for example, MₐP_{b}S_{c}O_{d});
MZPS (for example, MₐZn_{b}P_{c}S_{d});
MZPSO (for example, MₐZn_{b}P_{c}S_{d}Oₑ);
xM₂S-yP₂S₅;
xM₂S-yP₂S₅-zMX;
xM₂S-yP₂S₅-zP₂O₅;
xM₂S-yP₂S₅-zP₂O₅-wMX;
xM₂S-yM₂O-zP₂S₅;
xM₂S-yM₂O-zP₂S₅-wMX;
xM₂S-yM₂O-zP₂S₅-wP₂O₅;
xM₂S-yM₂O-zP₂S₅-wP₂O₅-vMX;
xM₂S-ySiS₂;
MPSX (for example, MₐPS_{c}X_{d} such as M₇P₃S₁₁X, M₇P₂S₈X, and M₆PS₅X);
MPSOX (for example, MₐP_{b}S_{c}O_{d}Xₑ);
MGPSX (for example, MₐGe_{b}P_{c}S_{d}Xₑ);
MGPSOX (for example, MₐGe_{b}P_{c}S_{d}OₑX_{f});
MSiPSX (for example, MₐSi_{b}P_{c}S_{d}Xₑ);
MSiPSOX (for example, MₐSi_{b}P_{c}S_{d}OₑX_{f});
MSnPSX (for example, MₐSn_{b}P_{c}S_{d}Xₑ);
MSnPSOX (for example, MₐSn_{b}P_{c}S_{d}OₑX_{f});
MZPSX (for example, MₐZn_{b}P_{c}S_{d}Xₑ);
MZPSOX (for example, MₐZn_{b}P_{c}S_{d}OₑX_{f});
M₃OX;
M₂HOX;
M₃PO₄;
M₃PS₄; and
MₐPO_{b}N_{c} (where a=2b+3c−5);
   wherein,
   M is an alkali metal ion, an alkaline earth metal ion, or a combination thereof, and wherein when M comprises an alkaline earth metal ion, then the number of M is adjusted to achieve electroneutrality;
   X is selected from F, Cl, Br, I, or a combination of at least two thereof;
   a, b, c, d, e, and f are numbers other than zero and are, independently in each formula, selected to achieve electroneutrality; and
   v, w, x, y, and z are numbers other than zero and are, independently in each formula, selected to obtain a stable compound.

For example, M can be selected from Li, Na, K, Rb, Cs, Be, Mg, Ca, Sr, Ba, or a combination of at least two thereof. According to a variant of interest, M is Li.

According to a variant of interest, the inorganic particles comprise an inorganic compound of formula MATP as herein defined.

According to another variant of interest, the inorganic particles comprise an argyrodite-type inorganic compound of formula Li₆PS₅X, wherein X is Cl, Br, I, or a combination of at least two thereof. For example, the inorganic particles can comprise an inorganic compound of formula Li₆PS₅Cl.

The inorganic particles can be present in the solid electrolyte at a concentration in the range from about 25 wt. % to about 95 wt. %, upper and lower limits included. For example, the inorganic particles can be present in the solid electrolyte at a concentration in the range from about 40 wt. % to about 90 wt. %, or from about 60 wt. % to about 90 wt. %, upper and lower limits included.

The mass ratio of “inorganic particles:ionic bifunctional molecule” can be in the range from 2:1 to 30:1, upper and lower limits included. For example, the mass ratio of “inorganic particles:ionic bifunctional molecule” can be in the range from 3:1 to 20:1, or from 5:1 to 15:1, upper and lower limits included.

The solid electrolyte as herein defined can further include a polymer. For example, the polymer may be selected for its compatibility with the various components of an electrochemical cell. Any known compatible polymer is contemplated. The polymer can be selected from linear or branched polymers. Non-limiting examples of polymers include polyethers (for example, a polyether based on poly(ethylene oxide) (PEO), poly(propylene oxide) (POP) or a combination of the two (such as an EO/PO copolymer)), polythioethers, polyesters, polythioesters, poly(dimethylsiloxanes), poly(alkylene carbonates), poly(alkylene thiocarbonates), poly(alkylene sulfones), poly(alkylene sulfamides), polyimides, polyamides, polyphosphazenes, polyurethanes, poly(vinyl alcohols), polyacrylonitriles, polyethacrylates and polymethacrylates, and copolymers thereof, optionally comprising crosslinked units derived from crosslinkable functional groups (such as acrylate, methacrylate, vinyl, glycidyl, mercapto functions, etc.) or crosslinked equivalents thereof.

According to an example, the polymer, if present in the electrolyte, can be the product of the reaction between at least one monomer comprising at least one polymerizable or crosslinkable functional group and a compound comprising at least one SH functional group.

According to another example, the polymer can be present in the solid electrolyte at a concentration in the range from about 0.1 wt. % to about 20 wt. %, upper and lower limits included. For example, the polymer can be present in the solid electrolyte at a concentration in the range from about 1 wt. % to about 15 wt. %, or from about 2 wt. % to about 10 wt. %, upper and lower limits included.

For example, the ionic bifunctional molecule as herein defined acts as a binder between the inorganic particles in the solid electrolyte as herein defined, whereby the binder can also further comprise the polymer as herein defined.

The solid electrolyte as herein defined can also optionally include an additive.

According to an example, the additive, if present in the electrolyte, can be a fluorinated compound comprising an amide function. The fluorinated compound can be of formula R⁶X⁶C(O)N(H)X⁷R⁷, where R⁶ and R⁷ are independently alkyl, cycloalkyl, heterocycloalkyl, aryl, or heteroaryl groups, X⁶ is O, NH, or is absent, and X⁷ is absent or is a C(O), S(O)₂, or Si(R⁸R⁹) group, where R⁸ and R⁹ are alkyl groups, and where at least one of R⁶, R⁷, R⁸, and R⁹ is a group substituted by one or more fluorine atoms. For example, R⁶ is a perfluorinated group and X⁶ is absent.

According to an example, the additive, if present in the electrolyte, can be present in the solid electrolyte at a concentration in the range from about 5 wt. % to about 40 wt. %, upper and lower limits included. For example, the additive can be present in the solid electrolyte at a concentration in the range from about 10 wt. % to about 35 wt. %, or from about 15 wt. % to about 30 wt. %, upper and lower limits included.

The present technology also relates to an electrochemical cell comprising a negative electrode, a positive electrode, and an electrolyte, wherein the electrolyte is as herein defined.

The positive electrode comprises a positive electrode material optionally on a current collector. The positive electrode material comprises a positive electrode electrochemically active material. Non-limiting examples of positive electrode electrochemically active materials include metal phosphates, lithiated metal phosphates, metal oxides, and lithiated metal oxides.

For example, the metal of the electrochemically active material can be selected from titanium (Ti), iron (Fe), magnesium (Mg), manganese (Mn), vanadium (V), nickel (Ni), cobalt (Co), aluminum (Al), chromium (Cr), copper (Cu), antimony (Sb) and a combination of at least two thereof, when compatible. According to a variant of interest, the metal of the electrochemically active material can be selected from titanium (Ti), iron (Fe), magnesium (Mg), manganese (Mn), vanadium (V), nickel (Ni), cobalt (Co), aluminum (Al), and a combination of at least two thereof, when compatible.

Non-limiting examples of positive electrode electrochemically active materials generally include metal phosphates and lithiated metal phosphates (for example, LiM′PO₄ and M′PO₄, where M′ is selected from Fe, Ni, Mn, Co, and a combination of at least two thereof), vanadium oxides and lithium vanadium oxides (for example, LiV₃O₈, V₂O₅, LiV₂O₅, and similar vanadium oxides and lithium vanadium oxides), and lithium metal oxides of formulae LiMn₂O₄, LiM″O₂ (where M″ is selected from Mn, Co, Ni, and a combination of at least two thereof) (such as NMC, LiMnₓCo_{y}Ni_{z}O₂ with x+y+z=1), Li(NiM′″)O₂ (where M″′ is selected from Mn, Co, Al, Fe, Cr, Ti, Zr, another similar metal, and a combination of at least two thereof), sulfur, elemental selenium, elemental iodine, iron(III) fluoride, copper(II) fluoride, lithium iodide, carbon-based active materials such as graphite, organic cathode active materials, or a combination of at least two of these electrochemically active materials, when compatible with each other.

The positive electrode material as herein defined can further include an electronically conductive material, a binder, a salt, an ionic bifunctional molecule (for example, an ionic bifunctional molecule as previously defined), and/or inorganic particles.

The negative electrode comprises a negative electrode electrochemically active material which is optionally on a current collector.

According to an example, the negative electrode electrochemically active material can comprise a metal film comprising an alkali or alkaline earth metal or an alloy comprising an alkali or alkaline earth metal. For example, the alkali metal can be selected from lithium and sodium.

According to another example, the negative electrode electrochemically active material can include an intermetallic compound (for example, SnSb, TiSnSb, Cu₂Sb, AlSb, FeSb₂, FeSn₂, and CoSn₂), a metal oxide, a metal nitride, a metal phosphide, a metal phosphate (for example, LiTi₂(PO₄)₃), a metal halide (for example, a metal fluoride), a metal sulfide, a metal oxysulfide, carbon (for example, graphite, graphene, reduced graphene oxide, hard carbon, soft carbon, exfoliated graphite, and amorphous carbon), silicon (Si), a silicon-carbon composite (Si—C), a silicon oxide (SiOₓ), a silicon oxide-carbon composite (SiOₓ—C), tin (Sn), a tin oxide-carbon composite (Sn—C), a tin oxide (SnOₓ), a tin oxide-carbon composite (SnOₓ—C), and a combination of at least two thereof, when compatible. For example, the metal oxide can be selected from compounds of formulae M″″_{b}O_{c} (where M″″ is Ti, Mo, Mn, Ni, Co, Cu, V, Fe, Zn, Nb, or a combination thereof, and b and c are numbers such that the ratio c:b is in the range from 2 to 3) for example, MoO₃, MoO₂, MoS₂, V₂O₅, and TiNb₂O₇), spinel oxides (for example, NiCo₂O₄, ZnCo₂O₄, MnCo₂O₄, CuCo₂O₄, and CoFe₂O₄) and LiM′″″O (where M′″″ is Ti, Mo, Mn, Ni, Co, Cu, V, Fe, Zn, Nb, or a combination thereof) (for example, lithium titanate (such as Li₄Ti₅O₁₂) or lithium molybdenum oxide (such as Li₂Mo₄O₁₃)).

According to another example, the negative electrode material can further comprise an electronically conductive material, a binder, a salt, an ionic bifunctional molecule (for example, an ionic bifunctional molecule as previously defined), and/or inorganic particles.

The present technology also relates to a battery comprising at least one electrochemical cell as herein defined. For example, said battery is selected from the group consisting of a lithium battery, a lithium-ion battery, a sodium battery, a sodium-ion battery, a potassium battery, a potassium-ion battery, a magnesium battery, and a magnesium-ion battery. According to a variant of interest, said battery is a lithium battery or a lithium-ion battery.

The presence of the ionic bifunctional molecule as herein defined in a solid electrolyte, for example, in an inorganic solid electrolyte or a polymer-ceramic hybrid solid electrolyte can significantly improve some of its physical and/or electrochemical properties.

According to an example, the presence of the ionic bifunctional molecule can, for example, substantially improve the mechanical strength of a solid electrolyte film and/or the densification of said solid electrolyte film after creep. According to another example, the presence of the ionic bifunctional molecule can substantially improve the ionic conductivity and/or electrochemical stability of the solid electrolyte film. In some cases, the presence of the ionic bifunctional molecule can also substantially improve the flammability safety of the solid electrolyte film.

### EXAMPLES

The following examples are for illustrative purposes and should not be construed as further limiting the scope of the invention as contemplated. These examples will be better understood by referring to the accompanying figures.

### Example 1—Preparation and Characterization of Bifunctional Ionic Salts

### a) Preparation of 1,1′-(1,6-hexamethylene) bis(1-methylpyrrolidinium) dibromide

8 g of 1-methylpyrrolidine (94.1 mmol), 10.4 g of 1,6-dibromohexane (42.8 mmol) and 20 ml of tetrahydrofuran (THF) were introduced into a 100 ml single-neck flask. The solution was heated at a temperature of about 50° C. for about 12 hours. The precipitate formed during the reaction was then recovered by filtration and washed three times with THF. The product thus obtained was dried under vacuum at a temperature of about 50° C. for about 24 hours.

### b) Preparation of 1,1′-(1,6-hexamethylene) bis(1-methylpyrrolidinium) bis(trifluoromethanesulfonyl)imide (Salt 1)

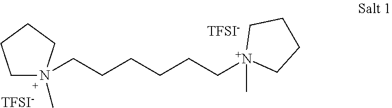

1,1′-(1,6-hexamethylene) bis(1-methylpyrrolidinium) bis(trifluoromethanesulfonyl)imide (Salt 1) was prepared by anionic exchange from lithium bis(trifluoromethanesulfonyl)imide (LiTFSI) and the 1,1′-(1,6-hexamethylene) bis(1-methylpyrrolidinium) dibromide prepared in Example 1(a). The anionic exchange was carried out in water at a temperature of about 40° C. for about 3 hours.

### c) Preparation of 1,1′-(1,12-dodecamethylene) bis(1-methylpyrrolidinium) dibromide

8 g of 1-methylpyrrolidine (94.1 mmol), 10.3 g of 1,12-dibromododecane (31.4 mmol) and 20 ml of THF were introduced into a 100 ml single-neck flask. The solution was heated to about 50° C. for about 12 hours. The precipitate formed during the reaction was then recovered by filtration and washed three times with diethyl ether. The product thus obtained was dried under vacuum at a temperature of about 50° C. for about 24 hours.

### d) Preparation of 1,1′-(1,12-dodecamethylene) bis(1-methylpyrrolidinium) bis(trifluoromethanesulfonyl)imide (Salt 2)

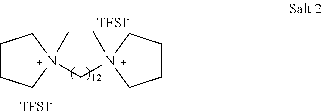

1,1′-(1,12-dodecamethylene) bis(1-methylpyrrolidinium) bis(trifluoromethanesulfonyl)imide (Salt 2) was prepared by anionic exchange from LiTFSI and the 1,1′-(1,12-dodecamethylene) bis(1-methylpyrrolidinium) dibromide prepared in Example 1(c). The anionic exchange was carried out in water at a temperature of about 40° C. for about 3 hours.

### e) Preparation of 1,1′-(2,2′-(ethylenedioxy)diethane) bis(1-methylpyrrolidinium) dichloride

4 g of 1-methylpyrrolidine (46.6 mmol), 2.9 g of 1,2-bis(2-chloroethoxy)ethane (15.6 mmol) and 10 ml of THF were introduced into a 50 ml single-neck flask. The solution was heated at a temperature of about 50° C. for about 48 hours. The precipitate formed during the reaction was then recovered by filtration and washed three times with diethyl ether. The product thus obtained was dried under vacuum at a temperature of about 50° C. for about 24 hours.

### f) Preparation of 1,1′-(2,2′-(ethylenedioxy)diethane) bis(1-methylpyrrolidinium) bis(trifluoromethanesulfonyl)imide (Salt 3)

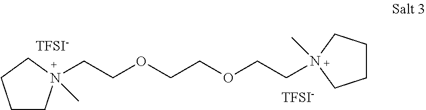

1,1′-(2,2′-(ethylenedioxy)diethane) bis(1-methylpyrrolidinium) bis(trifluoromethanesulfonyl)imide (Salt 3) was prepared by anionic exchange from LiTFSI and the 1,1′-(2,2′-(ethylenedioxy)diethane) bis(1-methylpyrrolidinium) dichloride prepared in Example 1(e). The anionic exchange was carried out in water at a temperature of about 40° C. for about 3 hours.

### g) Preparation of 1-(2-hydroxyethyl)-1-methylpyrrolidinium iodide

5.75 g of 1-(2-hydroxyethyl)pyrrolidine (50 mmol), 8.52 g of iodomethane (60 mmol) and 10 ml of THF were introduced into a 50 ml single-neck flask. The solution was heated at a temperature of about 50° C. for about 12 hours. The precipitate formed during the reaction was then recovered by filtration and washed three times with diethyl ether. The product thus obtained was dried under vacuum at a temperature of about 50° C. for about 24 hours.

### h) Preparation of 1-(2-chloroxyethyl)-1-methylpyrrolidinium iodide

5.14 g of the 1-(2-hydroxyethyl)-1-methylpyrrolidinium iodide prepared in Example 1(g) (20 mmol), 23.8 g of thionyl dichloride (0.2 mol) were introduced into a 50 ml single-neck flask. The solution was heated to a temperature of about 70° C. for about 24 hours. The product was precipitated in 100 ml diethyl ether. The precipitate was then recovered by filtration and washed three times with diethyl ether. The product thus obtained was dried under vacuum at a temperature of about 50° C. for about 24 hours.

### i) Preparation of 1,1′-(thiol bis(1,2-ethane)) bis(1-methylpyrrolidinum) iodide

3 g of the 1-(2-chloroxyethyl)-1-methylpyrrolidinium prepared in Example 1(h) (11 mmol), 0.43 g sodium sulfide (5.5 mmol), 0.04 g of sodium hydroxide (1 mmol), 40 ml of deionized water and 60 ml of methanol were introduced into a 250 ml single-neck flask. The solution was heated at a temperature of about 50° C. for about 24 hours. The precipitate formed during the reaction was then recovered by filtration and washed three times with diethyl ether. The product thus obtained was dried under vacuum at a temperature of about 50° C. for about 24 hours.

### j) Preparation of 1,1′-(thiol bis(1,2-ethane)) bis(1-methylpyrrolidinum) bis(trifluoromethanesulfonyl)imide (Salt 4)

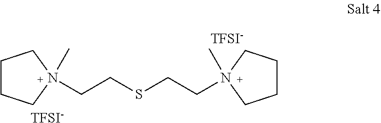

1,1′-(thiol bis(1,2-ethane)) bis(1-methylpyrrolidinum) bis(trifluoromethanesulfonyl)imide (Salt 4) was prepared by anionic exchange from LiTFSI and the 1,1′-(thiol bis(1,2-ethane)) bis(1-methylpyrrolidinum) iodide prepared in Example 1(i). The anionic exchange was carried out in a water:methanol mixture (2:8 by volume) at a temperature of about 40° C. for about 3 hours.

### k) Preparation of 3,3′-(1,6-hexamethylene) bis(1,2-dimethylimidazolium) dibromide

5.77 g of 1,2-dimethylimidazole (60 mmol), 4.88 g of 1,6-dibromohexane (20 mmol) and 10 ml of THF were introduced into a 50 ml single-neck flask. The solution was heated to a temperature of about 50° C. for about 12 hours. The precipitate formed during the reaction was then recovered by filtration and washed three times with diethyl ether. The product thus obtained was dried under vacuum at a temperature of about 50° C. for about 24 hours.

### l) Preparation of 3,3′-(1,6-hexamethylene) bis(1,2-dimethylimidazolium) bis(trifluoromethanesulfonyl)imide (Salt 5)

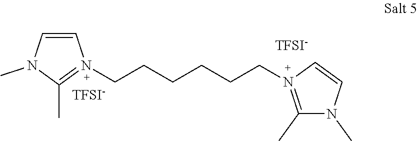

3,3′-(1,6-hexamethylene) bis(1,2-dimethylimidazolium) bis(trifluoromethanesulfonyl)imide (Sel 5) was prepared by anionic exchange from LiTFSI and the 3,3′-(1,6-hexamethylene) bis(1,2-dimethylimidazolium) dibromide prepared in Example 1(k). The anionic exchange was carried out in water at a temperature of about 40° C. for about 3 hours.

### Example 2—Nuclear Magnetic Resonance (NMR) Characterization

Salts 1 to 5 prepared in Example 1 were characterized by proton nuclear magnetic resonance (¹H NMR).

a) NMR characterization of Salt 1 prepared in Example 1(b)

The ¹H NMR spectrum of Salt 1 prepared in Example 1(b) was obtained in methanol-d4 (deuterated methanol or CD₃OD) as the solvent and the results obtained are shown in Table 1.

TABLE 1
¹H NMR results for Salt 1

| | Chemical shift |
|---|---|
| Type of proton | (δ ppm) |
| —CH₂—C₂H₄—N | 1.51 |
| —CH₂—CH₂—CH₂—N | 1.87 |
| Cyclic —CH₂—C₂H₄—CH₂—N | 2.21 |
| —CH₃ | 3.1 |
| —CH₂—CH₂—N | 3.39-3.45 |
| —CH₂—N—CH₂-cyclic | 3.54-3.60 |

### b) ¹H NMR Characterization of Salt 2 Prepared in Example 1(d)

The ¹H NMR spectrum of Salt 2 prepared in Example 1(d) was obtained in chloroform-d (deuterated chloroform or CDCl₃) as the solvent and the results obtained are presented in Table 2.

TABLE 2
¹H NMR results for Salt 2

| | Chemical shift |
|---|---|
| Type of proton | (δ ppm) |
| —CH₂—CH₂—CH₂—CH₂—C₂H₄—N | 1.17-1.50 |
| —CH₂—CH₂—N | 1.72 |
| Cyclic —CH₂—C₂H₄—CH₂—N | 2.20 |
| —CH₃ | 2.98 |
| —CH₂—N | 3.17-3.33 |
| —CH₂—N—CH₂-cyclic | 3.34-3.60 |

### c) ¹H NMR Characterization of Salt 3 Prepared in Example 1(f)

The ¹H NMR spectrum of Salt 3 prepared in Example 1(f) was obtained in CDCl₃ as the solvent and the results obtained are presented in Table 3.

TABLE 3
¹H NMR results for Salt 3

| | Chemical shift |
|---|---|
| Type of proton | (δ ppm) |
| Cyclic —CH₂—C₂H₄—CH₂—N | 2.08-2.33 |
| —CH₃ | 3.04 |
| —CH₂—O—CH₂—CH₂—N | 3.40-3.58 |
| —CH₂—N—CH₂-cyclic | 3.58-3.73 |
| —O—CH₂—CH₂—N | 3.81-3.92 |

### d) ¹H NMR Characterization of Salt 4 Prepared in Example 1(j)

The ¹H NMR spectrum of Salt 4 prepared in Example 1(j) was obtained in dimethyl sulfoxide-d6 (deuterated dimethyl sulfoxide or DMSO-d6) as the solvent and the results obtained are presented in Table 4.

TABLE 4
¹H NMR results for Salt 4

| | Chemical shift |
|---|---|
| Type of proton | (δ ppm) |
| Cyclic —CH₂—C₂H₄—CH₂—N | 2.11 |
| —CH₃ | 3.06 |
| —CH₂—N—CH₂-cyclic | 3.42-3.64 |
| —S—CH₂—CH₂—N | 3.71-3.87 |
| —S—CH₂—CH₂—N | 4.02-4.18 |

e) ¹H NMR characterization of Salt 5 prepared in Example 1(l)

The ¹H NMR spectrum of Salt 5 prepared in Example 1(l) was obtained in DMSO-d6 as the solvent and the results obtained are presented in Table 5.

TABLE 5
¹H NMR results for Salt 5

| | Chemical shift |
|---|---|
| Type of proton | (δ ppm) |
| —CH₂—C₂H₄—N | 1.19-1.38 |
| —CH₂—CH₂—N | 1.59-1.81 |
| Cyclic —CH₂—C₂H₄—N(═C(CH₃) | 2.57 |
| Cyclic —CH₂—C₂H₄—N—CH═CH—N(CH₃) | 3.76 |
| —CH₂—CH₂—N | 4.03-4.16 |
| Cyclic —CH₂—C₂H₄—N—CH═CH—N(CH₃) | 7.62 |

### Example 3—Thermal and Thermogravimetric Analyses

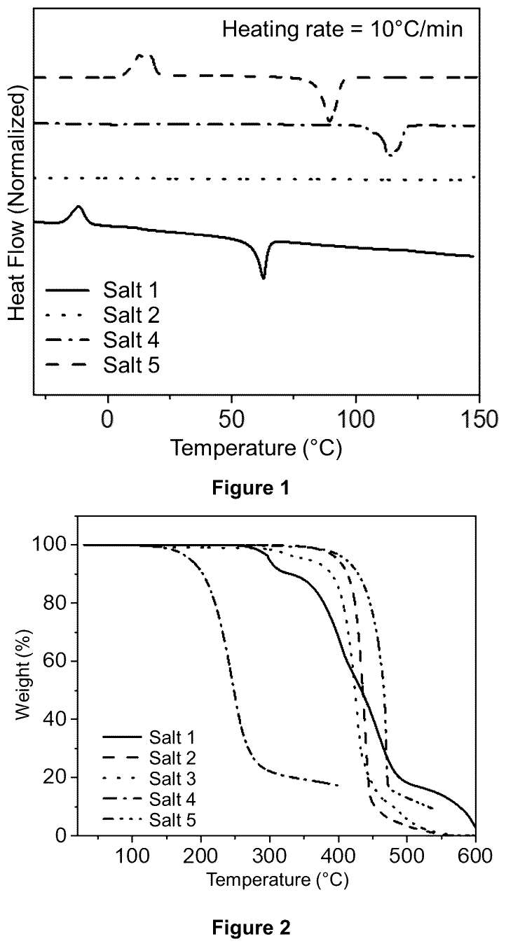

FIG. 1 presents the Differential Scanning Calorimetry (DSC) analysis results obtained for Salts 1, 2, 4 and 5 respectively prepared in Examples 1(b), 1(d) and 1(l). DSC analysis was carried out over a temperature range from about −20° C. to about 148° C. at a heating rate (or speed) of 10° C./min. As shown in FIG. 1, Salt 1 has a crystallization temperature of −11° C. and a melting temperature of 63° C.

FIG. 2 presents the thermogravimetric analysis (TGA) results obtained for Salts 1 to 5 respectively prepared in Examples 1(b), 1(d), 1(f), 1(j) and 1(l). Thermogravimetric analysis was carried out over a temperature range from about 40° C. to about 600° C. As shown in FIG. 2, Salt 1 has a decomposition point of around 295° C.

The results of the thermal and thermogravimetric analyses are presented in Table 6.

TABLE 6
DSC and TGA results obtained for Salts 1 to 5

| | Crystallization | Melting point | Decomposition |
|---|---|---|---|
| Salt | point (° C.) | (° C.) | point (° C.) |
| Salt 1 | −11 | 63 | 295 |
| Salt 2 | ND | ND | 356 |
| Salt 3 | ND | ND | 298 |
| Salt 4 | 64 | 114 | 164 |
| Salt 5 | 18 | 89 | 364 |

*ND: not detectable between about −50° C. and about 150° C.

### Example 4—Chemical and Electrochemical Stability

The electrochemical stability of a liquid electrolyte comprising Salt 1 prepared in Example 1(b) was characterized by linear sweep voltammetry (LSV) and cyclic voltammetry (CV).

### a) Cell Configurations for Electrochemical Stability Analyses

A liquid electrolyte comprising LiTFSI, TEGDME as the solvent and Salt 1 prepared in Example 1(b) was prepared. A liquid electrolyte comprising LiTFSI and TEGDME and a liquid electrolyte comprising LiTFSI, TEGDME and [PYR_{1,4}]TFSI were also prepared for comparison. The composition of the liquid electrolytes used for electrochemical stability analyses is presented in Table 7.

TABLE 7
Liquid electrolyte compositions

| | Lithium salt | Solvent | Ionic salt | |
|---|---|---|---|---|
| | LiTFSI | TEGDME | [PYR_{1,4}]TFSI | Salt 1 |
| Electrolyte | (% by weight) | (% by weight) | (% by weight) | (% by weight) |
| E1 (comparative) | 19% | 81% | — | — |
| E2 (comparative) | 11.9% | 36.2% | 51.8% | — |
| E3 | 11.9% | 36.2% | — | 51.8% |

Celgard™ 2325 separators made of a three-layer microporous polypropylene-polyethylene-polypropylene (PP/PE/PP) membrane with a thickness of about 25 μm were impregnated with the above liquid electrolytes. Discs with a diameter of 16 mm were then cut from the membranes impregnated with liquid electrolyte.

Cells for electrochemical stability analyses were assembled according to the following procedure. The cells were assembled in a button cell configuration. The disks impregnated with liquid electrolyte prepared in the present example were placed and pressed between an aluminum and a lithium electrode for the oxidation process (Al/electrolyte/Li) and between a copper and a lithium electrode for the reduction process (Cu/electrolyte/Li).

The configuration of each cell is presented below:
Cell 1: Electrode/E1/Electrode
Cell 2: Electrode/E2/Electrode
Cell 3: Electrode/E3/Electrode

### b) Electrochemical Stability Analyses

Electrochemical stability measurements for Cells 1 to 3 assembled in Example 4(a) were carried out by LSV. Electrochemical stability measurements for cells comprising electrolytes E2 and E3 were also carried out by CV. Measurements were performed with a Bio-Logic™ VMP-300 system at a scan rate of 0.1 mV/s.

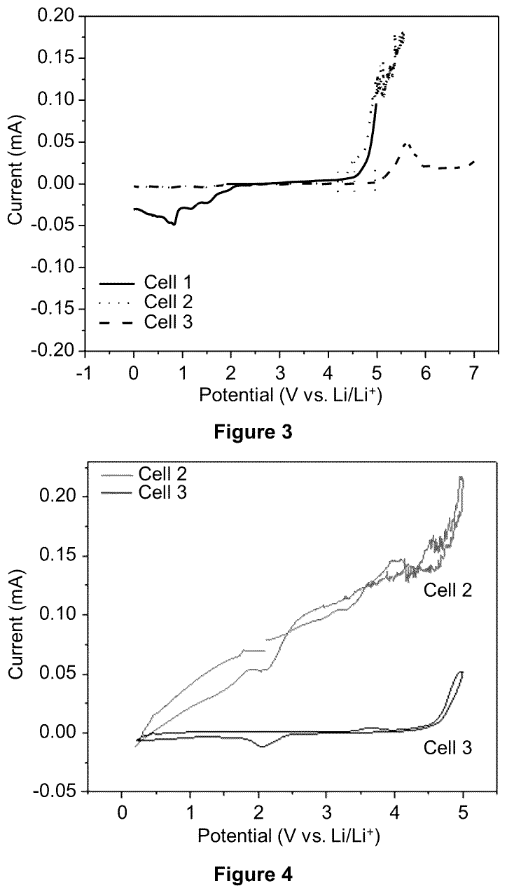

FIGS. 3 and 4 show the results of the LSV and CV analysis respectively. As shown in FIGS. 3 and 4, Cell 3 comprising the liquid electrolyte comprising LiTFSI, TEGDME and Salt 1 prepared in Example 1(b) has an electrochemical stability greater than Cell 2 comprising the liquid electrolyte comprising LiTFSI, TEGDME and [PYR_{1,4}]TFSI.

### c) Chemical Stability Analysis

The chemical stability of TEGDME, a solution comprising [PYR_{1,4}]TFSI in TEGDME and a solution comprising Salt 1 prepared in Example 1(b) in TEGDME towards lithium metal was analyzed.

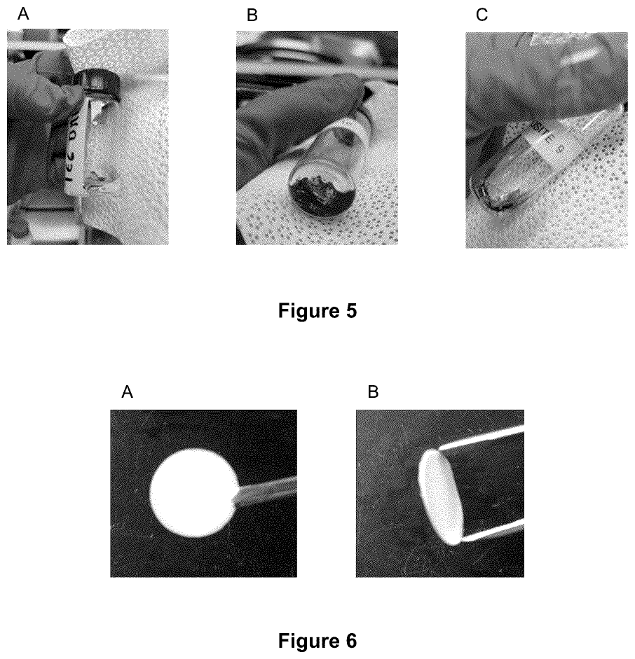

FIG. 5 presents images of lithium foils dipped respectively in (A) TEGDME, in (B) a solution comprising [PYR_{1,4}]TFSI in TEGDME in a ratio TEGDME: [PYR_{1,4}]TFSI (40:60 by weight), and in (C) a solution comprising Salt 1 prepared in Example 1(b) in TEGDME in a ratio TEGDME:Salt 1 (41:59 by weight). Lithium foils were submerged in the three different solutions for about one week. As shown in FIG. 5, only the solution comprising [PYR_{1,4}]TFSI in TEGDME changed color from transparent to black (FIG. 5(B)). This indicates that the chemical stability of TEGDME and the solution comprising Salt 1 in TEGDME is greater than that of the solution comprising [PYR_{1,4}]TFSI in TEGDME.

### Example 5—Preparation and Characterization of an Inorganic Solid Electrolyte

### a) Preparation of Inorganic Solid Electrolyte Pellets

0.294 g of Li_{1.3}Al_{0.3}Ti_{1.7}(PO₄)₃ (LATP, Toshima™), 0.126 g of N-methyltrifluoroacetamide (NMTFAm) and 0.06 g of Salt 1 prepared in Example 1(b) were thoroughly mixed and ground in a mortar at room temperature to obtain a solid electrolyte powder. Round pellets with a diameter of about 16 mm and a thickness of about 900 μm were obtained by compressing the solid electrolyte powder under a pressure of 120 psi.

FIG. 6 presents images of a solid electrolyte pellet showing respectively in (A) its diameter (about 16 mm), and in (B) its thickness (about 900 μm).

### b) Ionic Conductivity

The ionic conductivity of the inorganic solid electrolyte pellets prepared in Example 5(a) was characterized by electrochemical impedance spectroscopy.

To achieve this, the inorganic solid electrolyte pellets prepared in Example 5(a) were placed and pressed between two stainless steel electrodes.

Electrochemical impedance spectroscopy measurements were carried out using a Bio-Logic™ VMP-300 system with an amplitude of 100 mV over a frequency range from 1 MHz to 200 mHz. An ionic conductivity of 2.5 mS/cm was measured at a temperature of 60° C.

### Example 6—Preparation and Characterization of Ceramic-Ionic Plastic Salt Composite Solid Electrolyte Films

The crosslinkable polymer used in the following example is a multi-branched polyether comprising crosslinkable units as described in U.S. Pat. No. 7,897,674 (hereinafter referred to as the “U.S. '674 polymer”).

The ionic plastic crystal used in the following example is an ionic plastic crystal including a delocalized bis(trifluoromethanesulfonyl)imide [TFSI]⁻ anion paired with a cation derived from 1,8-diazabicyclo[5.4.0]undec-7-ene (DBU), as described in PCT patent application published under number WO 2022/165598 (hereinafter referred to as the “WO '598 plastic crystal”).

### a) Preparation of Ionic Ceramic-Salt Composite Solid Electrolyte Films

Composite solid electrolyte films comprising a sulfide-based ceramic and Salt 1 prepared in Example 1(b) were prepared. Composite solid electrolyte films comprising a sulfide-based ceramic and the WO '598 plastic crystal were also prepared for comparison purposes.

All manipulations were carried out in a glovebox under an argon atmosphere (0.1 ppm H₂O; 0.1 ppm O₂).

Two sizes (about 3 μm and less than 1 μm) of sulfide-based ceramic inorganic solid electrolyte particles (Li₆PS₅Cl) were mixed in a mass ratio of 90:10 or 75:25 using a vortex. The binder used comprises a 40:60 by weight mixture of the U.S. '674 polymer with 0.5 wt. % of UV crosslinker and the WO '598 plastic crystal or one of Salts 1 to 5 was dissolved in dichloromethane (DCM). For binders comprising Salts 4 and 5, a small amount of acetone was added in order to achieve good dissolution of the salt.

The mass ratio of sulfide to binder was 90:10 by weight. The amount of DCM or DCM and acetone was adjusted to obtain a mixture with an appropriate viscosity. The mixture thus obtained was coated onto a previously degreased aluminum foil. The film thus obtained was dried in a glovebox. After drying, UV curing was carried out for about 15 seconds.

The composition of the ceramic-ionic plastic composite solid electrolyte films is presented in Table 8.

TABLE 8
Composition of ceramic-ionic plastic salt composite solid electrolyte films

| | | Binder | | |
|---|---|---|---|---|
| | Mass | US′674 polymer | | |
| | proportion of | with 0.5 wt. % of | WO′598 | |
| | Li₆PS₅Cl | UV crosslinker | plastic crystal | Salt |
| | particles | (% by weight in | (% by weight in | (% by weight in |
| Electrolyte | (3 μm:<1 μm) | the binder) | the binder) | the binder) |
| E4 | 90:10 | 40% | 60% | — |
| (comparative) | | | | |
| E5 | 75:25 | 40% | 60% | — |
| (comparative) | | | | |
| E6 | 90:10 | 40% | — | Salt 1 (60%) |
| E7 | 75:25 | 40% | — | Salt 1 (60%) |
| E8 | 90:10 | 40% | — | Salt 2 (60%) |
| E9 | 90:10 | 40% | — | Salt 3 (60%) |
| E10 | 90:10 | 40% | — | Salt 4 (60%) |
| E11 | 90:10 | 40% | — | Salt 5 (60%) |

### b) Ionic Conductivity of Ceramic-Ionic Plastic Salt Composite Solid Electrolyte Films

Pellets of 10 mm in diameter were taken from the ceramic-ionic plastic salt composite solid electrolyte films prepared in Example 6(a). The pellets were placed in a 10 mm diameter mold and compressed under a pressure of 2.8 tons using a press. The pellets were then placed in a conductivity cell at a pressure of 5 MPa closed under an inert argon atmosphere. The configuration of each cell is presented below:
Cell 4: Electrode/E4/Electrode
Cell 5: Electrode/E5/Electrode
Cell 6: Electrode/E6/Electrode
Cell 7: Electrode/E7/Electrode
Cell 8: Electrode/E8/Electrode
Cell 9: Electrode/E9/Electrode
Cell 10: Electrode/E10/Electrode
Cell 11: Electrode/E11/Electrode

The ionic conductivity measurements of the cells assembled in the present example were carried out with a VMP-300 multi-channel potentiostat (Bio-Logic™). Measurements were carried out over a frequency range from 7 MHz to 200 mHz at an amplitude of 50 mV over a temperature range from −10° C. to 70° C. (in increase every 10° C.) and over a temperature range from 70° C. to 20° C. (in decrease every 10° C.).

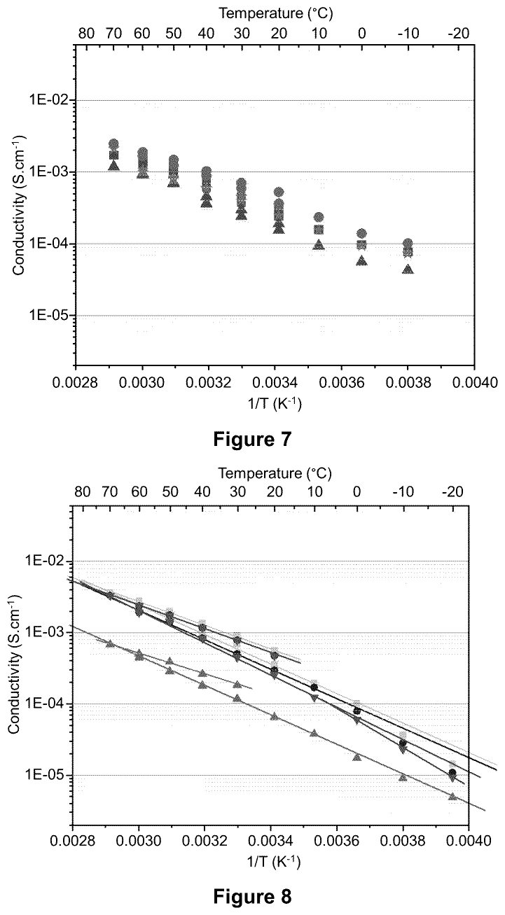

Impedance measurements were obtained after a stabilization period of about one hour. Two impedance measurements were recorded at each temperature with 15 minutes between each measurement. FIG. 7 presents the measured ionic conductivity results as a function of the temperature for Cells 4 ( [symbol] ), 5 (▴), 6 (●), and 7 (★). FIG. 8 presents the measured ionic conductivity results as a function of temperature for Cells 8 ( [symbol] ), 9 (●), 10 (▴), and 11 (▾).

It is possible to observe in FIG. 7 that the ionic conductivity of Cells 5 and 7 is lower than that of Cells 4 and 6 respectively comprising mass ratios of Li₆PS₅Cl (3 μm: <1 μm) of 75:25 and 90:10.

FIG. 7 shows that the ionic conductivity of Cells 6 and 7 is substantially higher than that of Cells 4 and 5 comprising ceramic-ionic plastic salt composite solid electrolyte films including respectively Salt 1 and the WO '598 plastic crystal. This indicates a better interaction of the lithium ions from the sulfide-based ceramic type inorganic solid electrolytes with the bifunctional ionic salt.

FIG. 7 also shows that the ionic conductivity of Cell 7 (Li₆PS₅Cl mass ratio (3 μm: <1 μm) of 75:25) including Salt 1 is similar to that of Cell 4 (Li₆PS₅Cl mass ratio (3 μm: <1 μm) of 90:10) with the WO '598 plastic crystal. This indicates that the bifunctional ionic salt allows increasing the addition of Li₆PS₅Cl particles of smaller size (<1 μm) and thus to obtain under compression a better compactness of the ceramic-ionic plastic salt composite solid electrolyte film while maintaining substantially high performance.

At a temperature of 20° C., the ionic conductivity results for Cells 6 and 7 are slightly lower than those obtained for sulfide-based ceramic type inorganic solid electrolyte particles (Li₆PS₅Cl) compressed alone and without an aluminum support, but measured under the same conditions.

It is possible to observe in FIG. 8 that the ionic conductivities obtained for Cells 8, 9 and 11 comprising ceramic-ionic plastic salt composite solid electrolyte films including respectively Salts 2, 3 and 5 are higher than that obtained for Cell 10 comprising a ceramic-ionic plastic salt composite solid electrolyte film including Salt 4.

### c) Scanning Electron Microscopy (SEM) Characterization of Ceramic-Ionic Plastic Salt Composite Solid Electrolyte Films

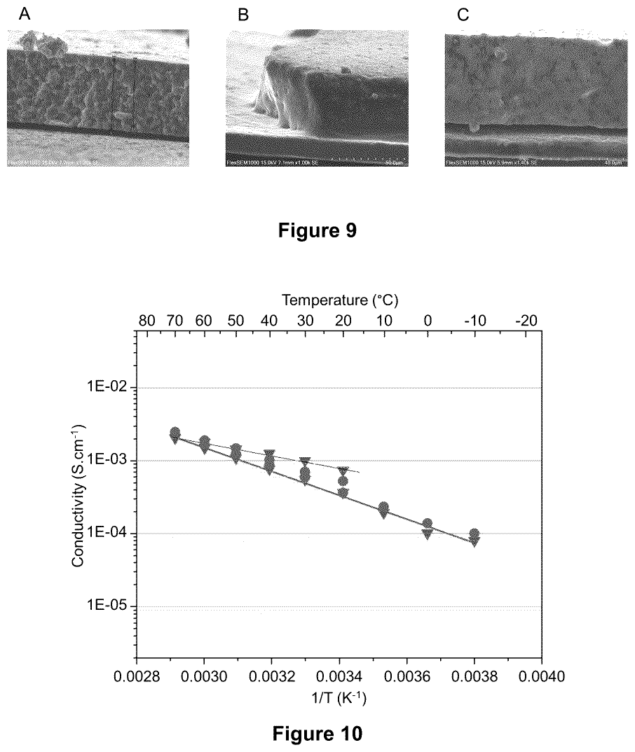

FIG. 9 shows SEM images obtained in (A) before creep, and in (B) and (C) after creep at a temperature of about 70° C. for the E6 ceramic-ionic plastic salt composite solid electrolyte film prepared in Example 6(a).

FIG. 9(A) shows that after compression, but before creep, the ceramic-ionic plastic salt composite solid electrolyte film is substantially dense and has a thickness of about 40 μm. It is possible to distinguish individual particles and/or agglomerates of sulfide-based ceramics.

FIGS. 9(B) and (C) show the effect of creep at a temperature of 70° C. (above the melting temperature of Salt 1) and down to room temperature. It is possible to observe that after creep, the ceramic-ionic plastic salt composite solid electrolyte film is substantially denser and no longer exhibits agglomerate. This can be useful in so-called “all-solid” configuration, especially in lithium metal configuration in order to resist lithium dendrites.

### Example 7—Preparation and Characterization of Ceramic-Ionic Plastic Salt Composite Solid Electrolyte Films (4,4′-Thiobisbenzenethiol (TBT) Crosslinking)

### a) Preparation of Ceramic-Ionic Plastic Salt Composite Solid Electrolyte Films and TBT Crosslinking

All manipulations were carried out in a glovebox under an argon atmosphere (0.1 ppm H₂O; 0.1 ppm O₂).

Two sizes (about 3 μm and less than 1 μm) of sulfide-based ceramic type inorganic solid electrolyte particles (Li₆PS₅Cl) were mixed in a mass ratio of 90:10 using a vortex.

The binder used comprises a 40:60 by weight mixture of the U.S. '674 polymer with 4.0 wt. % TBT and Salt 1 prepared in Example 1(b) dissolved in DCM.

The mass ratio of sulfide to binder was 90:10 by weight. The amount of DCM was adjusted to obtain a mixture with an appropriate viscosity. The mixture thus obtained was coated onto a previously degreased aluminum foil. The film thus obtained was dried in a glove box.

The composition of the ceramic-ionic plastic salt composite solid electrolyte films is presented in Table 9.

TABLE 9
Composition of ceramic-ionic plastic salt composite solid electrolyte films

| | Mass proportion | Binder | |
|---|---|---|---|
| | of Li₆PS₅Cl | Polymer | Salt |
| | particles | (% by weight | (% by weight |
| Electrolyte | (3 μm:<1 μm) | in the binder) | in the binder) |
| E12 | 90:10 | US′674 without TBT | Salt 1 (60%) |
| (comparative) | | 40% | |
| E13 | 90:10 | US′674 with 4.0 wt. % TBT | Salt 1 (60%) |
| | | 40% | |

### b) Ionic Conductivity of Polymer-Ceramic Hybrid Solid Electrolyte Films

Pellets of 10 mm in diameter were taken from the ceramic-ionic plastic salt composite solid electrolyte films prepared in Example 7(a). The pellets were placed in a 10 mm diameter mold and compressed under a pressure of 2.8 tons using a press. The pellets were then placed in a conductivity cell at a pressure of 5 MPa closed under an inert argon atmosphere.

The configuration of each cell is presented below:
Cell 12: Electrode/E12/Electrode
Cell 13: Electrode/E13/Electrode

The ionic conductivity measurements of the cells assembled in the present example were carried out with a VMP-300 multi-channel potentiostat (Bio-Logic™). The measurements were carried out over a frequency range from 7 MHz to 200 mHz at an amplitude of 50 mV over a temperature range from −10° C. to 70° C. (in increase every 10° C.) and over a temperature range from 70° C. to 20° C. (in decrease every 10° C.).

Impedance measurements were obtained after a stabilization period of about one hour. Two impedance measurements were recorded at each temperature with 15 minutes between each measurement. FIG. 10 presents the measured ionic conductivity results as a function of temperature for Cells 12 (e) and 13 (T).

The crosslinking of the U.S. '674 polymer via the insertion of TBT between the chains of the U.S. '674 polymer allows inhibiting the ionic conduction of lithium ions through the U.S. '674 polymer, and therefore makes it possible to significantly increase the ionic conduction of ceramic-ionic plastic salt composite solid electrolyte films, particularly after the creep of Salt 1. This allows confirming the interaction between ionic plastic salt and sulfide-based ceramics (such as Li₆PS₅Cl), as well as the positive effect of the creep of the ionic plastic salt on the density of the resulting film and on its ionic conductivity. The ionic conductivity of the ceramic-ionic plastic salt-TBT composite solid electrolyte film is substantially identical to that obtained for a sulfide-based ceramic type inorganic solid electrolyte film (Li₆PS₅Cl).

Several modifications could be made to any of the above-described embodiments without departing from the scope of the present invention as contemplated. The references, patents or documents of scientific literature referred to in the present application are incorporated herein by reference in their entirety and for all purposes.

What is claimed is:

1. A solid electrolyte comprising inorganic particles and an ionic bifunctional molecule of Formulae I or II: 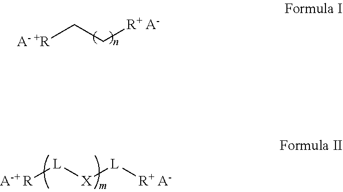
wherein,
A⁻ is a delocalized anion;
R⁺ is selected from —N⁺(R₁R₂R₃) and —P⁺(R₁R₂R₃) groups excluding cations derived from amidine, guanidine and phosphazene superbases;
R₁, R₂, and R₃ are independently selected from a substituted or unsubstituted linear or branched C₁₋₁₂alkyl group; or R₁ and R₂ together with the nitrogen or phosphorus atom form a heterocycle having one or more rings and having from 3 to 12 members and R₃ is as previously defined; or R₁, R₂, and R₃ together with the nitrogen or phosphorus atom form a partially unsaturated heterocycle or heteroaromatic having one or more rings and having from 5 to 12 members;
L is a linear or branched C₂₋₄alkylene;
X is O or S;
m is a number in the range from 1 to 6; and
n is a number in the range from 1 to 11.

2. The solid electrolyte of claim 1, wherein the delocalized anion is selected from hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), 2-trifluoromethyl-4,5-dicyanoimidazolate (TDI⁻), 4,5-dicyano-1,2,3-triazolate (DCTA⁻), bis(pentafluoroethylsulfonyl)imide (BETI⁻), difluorophosphate (DFP⁻), tetrafluoroborate (BF₄⁻), bis(oxalato)borate (BOB⁻), nitrate (NO₃⁻), perchlorate (ClO₄⁻), hexafluoroarsenate (AsF₆⁻), trifluoromethanesulfonate (CF₃SO₃⁻ or ⁻OTf), fluoroalkylphosphate ([PF₃(CF₂CF₃)₃]⁻ or FAP⁻), tetrakis(trifluoroacetoxy)borate ([B(OCOCF₃)₄]⁻ or TFAB⁻), bis(1,2-benzenediolato(2-)-O,O′)borate ([B(C₆O₂)₂]⁻ or BBB⁻), difluoro(oxalato)borate (BF₂(C₂O₄)⁻ or FOB⁻), and an anion of formula BF₂O₄R^{x} (R^{x}=C₂₋₄alkyl), preferably the delocalized anion is selected from hexafluorophosphate (PF₆⁻), bis(trifluoromethanesulfonyl)imide (TFSI⁻), bis(fluorosulfonyl)imide (FSI⁻), (fluorosulfonyl)(trifluoromethanesulfonyl)imide (FTFSI⁻), tetrafluoroborate (BF₄⁻), and trifluoromethanesulfonate (CF₃SO₃⁻ or ⁻OTf) and more preferably the delocalized anion is bis(trifluoromethanesulfonyl imide (TFSI⁻).

3-4. (canceled)

5. The solid electrolyte of claim 1, wherein R⁺ is an —N^{m}(R₁R₂R₃) group.

6. The solid electrolyte of claim 5, wherein:
(i) R₁, R₂, and R₃ are independently selected from substituted or unsubstituted linear or branched C₁₋₁₂alkyl groups,
(ii) R₁, R₂, and R₃, are independently selected from linear or branched C₁₋₁₂alkyl groups, or at least one of R₁, R₂, or R₃ is substituted by a halogen atom or an alkoxyl, ether, ester, or siloxy group,
(iii) R₁ and R₂ together with the nitrogen atom form a heterocycle having one or more rings and having from 3 to 12 members and R₃ is as defined in claim 1, preferably R₃ is a C₁₋₁₂ alkyl or a C₁₋₄alkyl; or
(iv) R₁, R₂, and R₃ together with the nitrogen atom form a partially unsaturated heterocycle or heteroaromatic having one or more rings and having from 5 to 12 members.

7-9. (canceled)

10. The solid electrolyte of claim 5, wherein R⁺ is selected from: 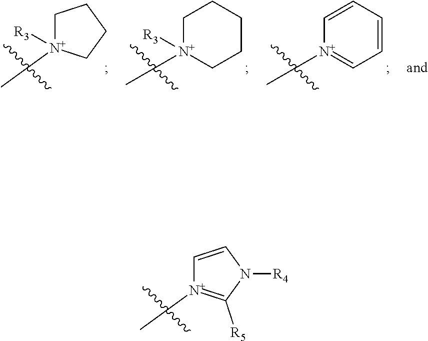
wherein,
R₃ is as defined in claim 1, and preferably R₃ is an unsubstituted C₁₋₄alkyl group preferably selected from a methyl group, an ethyl group, an n- or i-propyl group, and an n-, i-, s-, or t-butyl group;
R₄ is a substituted or unsubstituted linear or branched C₁₋₁₂alkyl, C₁₋₁₂alkenyl, or C₁₋₁₂alkynyl group, and preferably R₄ is a C₁₋₁₂alkyl group;
R₅ is a hydrogen atom, or a substituted or unsubstituted linear or branched C₁₋₁₂alkyl, C₁₋₁₂alkenyl, or C₁₋₁₂alkynyl group, and preferably R₅ is a C₁₋₄alkyl group; and
the heterocycle is optionally substituted.

11-14. (canceled)

15. The solid electrolyte of claim 1, wherein R⁺ is a —P⁺(R₁R₂R₃) group.

16. The solid electrolyte of claim 15, wherein:
(i) R₁, R₂, and R₃ are independently selected from substituted or unsubstituted linear or branched C₁₋₁₂alkyl groups; or
(ii) R₁, R₂, and R₃, are independently selected from linear or branched C₁₋₁₂alkyl groups, or at least one of R₁, R₂, or R₃ is substituted by a halogen atom or an alkoxyl, ether, ester, or siloxy group.

17. (canceled)

18. The solid electrolyte of claim 1, wherein n is a number in the range from 2 to 10, or from 3 to 8, or from 4 to 6.

19. The solid electrolyte of claim 1, wherein the ionic bifunctional molecule is selected from the group consisting of 1,1′-(1,6-hexamethylene) bis(1-methylpyrrolidinium) bis(trifluoromethanesulfonyl)imide, 1,1′(1,12-dodecamethylene) bis(1-methyl pyrrolidinium bis(trifluoromethanesulfonyl)imide, 1,1′-(2,2′-(ethylenedioxy)diethane)bis(1-methylpyrrolidinium) bis(trifluoromethanesulfonyl)imide, 1,1′-(thiol bis(1,2-ethane) bis(1-methylpyrrolidinum) bis(trifluoromethanesulfonyl)imide, and 3,3′-(1,6-hexamethylene) bis(1,2-dimethylimidazolium) bis(trifluoromethanesulfonyl)imide.

20-23. (canceled)

24. The solid electrolyte of claim 1, wherein the ionic bifunctional molecule is present at a concentration of from about 0.5 wt. % to about 50 wt. %, or from about 2 wt. % to about 30 wt. %, or from about 4 wt. % to about 20 wt. %, or from about 5 wt. % to about 15 wt. % in the solid electrolyte.

25. The solid electrolyte of claim 1, wherein the inorganic particles comprise a material selected from glasses, glass-ceramics, ceramics, nanoceramics, and a combination of at least two thereof.

26-27. (canceled)

28. The solid electrolyte of claim 25, wherein the inorganic particles comprise:
(i) a fluoride-, phosphide-, sulfide-, oxysulfide- or oxide-based ceramic, glass, or glass-ceramic;
(ii) a LISICON, thio-LISICON, argyrodite, garnet, NASICON, perovskite, oxide, sulfide, oxysulfide, phosphide or fluoride compound in crystalline and/or amorphous form, or a combination of at least two thereof;
(iii) a compound selected from inorganic compounds of formulae MLZO (for example, M₇La₃Zr₂O₁₂, M₍₇₋ₐ₎La₃Zr₂Al_{b}O₁₂, M₍₇₋ₐ₎La₃Zr₂Ga_{b}O₁₂, M₍₇₋ₐ₎La₃Zr_{(2-b)}Ta_{b}O₁₂, and M₍₇₋ₐ₎La₃Zr_{(2-b)}Nb_{b}O₁₂); MLTaO (for example, M₇La₃Ta₂O₁₂, M₅La₃Ta₂O₁₂, and M₆La₃Ta_{1.5}Y_{0.5}O₁₂); MLSnO (for example, M₇La₃Sn₂O₁₂); MAGP (for example, M₁₊ₐAlₐGe₂₋ₐ(PO₄)₃); MATP (for example, M₁₊ₐAlₐTi₂₋ₐ(PO₄)₃); MLTiO (for example, M₃ₐLa_{(2/3-a)}TiO₃); MZP (for example, MₐZr_{b}(PO₄)_{c}); MCZP (for example, MₐCa_{b}Zr_{c}(PO₄)_{d}); MGPS (for example, MₐGe_{b}P_{c}S_{d} such as M₁₀GeP₂S₁₂); MGPSO (for example, MₐGe_{b}P_{c}S_{d}Oₑ); MSiPS (for example, MₐSi_{b}P_{c}S_{d} such as M₁₀SiP₂S₁₂); MSiPSO (for example, MₐSi_{b}P_{c}S_{d}Oₑ); MSnPS (for example, MₐSn_{b}P_{c}S_{d} such as M₁₀SnP₂S₁₂); MSnPSO (for example, MₐSn_{b}P_{c}S_{d}Oₑ); MPS (for example, MₐP_{b}S_{c} such as M₇P₃S₁₁); MPSO (for example, MₐP_{b}S_{c}O_{d}); MZPS (for example, MₐZn_{b}P_{c}S_{d}); MZPSO (for example, MₐZn_{b}P_{c}S_{d}Oₑ); xM₂S-yP₂S₅; xM₂S-yP₂S₅-zMX; xM₂S-yP₂S₅-zP₂O₅; xM₂S-yP₂S₅-zP₂O₅-wMX; xM₂S-yM₂O-zP₂S₅; xM₂S-yM₂O-zP₂S₅-wMX; xM₂S-yM₂O-zP₂S₅-wP₂O₅; xM₂S-yM₂O-zP₂S₅-wP₂O₅-vMX; xM₂S-ySiS₂; MPSX (for example, MₐPS_{c}X_{d} such as M₇P₃S₁₁X, M₇P₂S₈X, and M₆PS₅X); MPSOX (for example, MₐP_{b}S_{c}O_{d}Xₑ); MGPSX (MₐGe_{b}P_{c}S_{d}Xₑ); MGPSOX (MₐGe_{b}P_{c}S_{d}OₑX_{f}); MSiPSX (MₐSi_{b}P_{c}S_{d}Xₑ); MSiPSOX (MₐSi_{b}P_{c}S_{d}OₑX_{f}); MSnPSX (MₐSn_{b}P_{c}S_{d}Xₑ); MSnPSOX (MₐSn_{b}P_{c}S_{d}OₑX_{f}); MZPSX (MₐZn_{b}P_{c}S_{d}Xₑ); MZPSOX (MₐZn_{b}P_{c}S_{d}OₑX_{f}); M₃OX; M₂HOX; M₃PO₄; M₃PS₄; and MₐPO_{b}N_{c} (where a=2b+3c−5);
wherein,
M is an alkali metal ion, an alkaline earth metal ion, or a combination of at least two thereof, and wherein when M comprises an alkaline earth metal ion, then the number of M is adjusted to achieve electroneutrality, preferably M is selected from Li, Na, K, Rb, Cs, Be, Mg, Ca, Sr, Ba, or a combination of at least two thereof, and more preferably M is Li;
X is selected from F, Cl, Br, I, or a combination of at least two thereof;
a, b, c, d, e, and f are numbers other than zero and are, independently in each formula, selected to achieve electroneutrality; and
v, w, x, y, and z are numbers other than zero and are, independently in each formula, selected to obtain a stable compound;
(iv) an inorganic compound of formula MATP;
(v) an argyrodite-type inorganic compound of formula Li₆PS₅X, wherein X is C, Br, I, or a combination of at least two thereof; or
(vi) an inorganic compound of formula Li₆PS₅Cl.

29-33. (canceled)

34. The solid electrolyte of claim 1, wherein the inorganic particles are present at a concentration of from about 25 wt. % to about 95 wt. %, or from about 40 wt. % to about 90 wt. %, or from about 60 wt. % to about 90 wt. % in the solid electrolyte.

35. The solid electrolyte of claim 1, wherein the ratio “inorganic particles:ionic bifunctional molecule” by weight is in the range from 2:1 to 30:1, or from 3:1 to 20:1, or from 5:1 to 15:1.

36. The solid electrolyte of claim 1, which further comprises a polymer.

37. The solid electrolyte of claim 36, wherein the polymer is:
(i) the reaction product of at least one monomer comprising at least one polymerizable or crosslinkable function and a compound comprising at least one SH functional group; or
(ii) a linear or branched polymer selected from polyethers, polythioethers, polyesters, polythioesters, poly(dimethylsiloxanes), poly(alkylene carbonate), poly(alkylene thiocarbonate), poly(alkylene sulfones), poly(alkylene sulfamides), polyimides, polyamides, polyphosphazenes, polyurethanes, poly(vinyl alcohols), polyacrylonitriles, polyethacrylates and polymethacrylates, and copolymers thereof, preferably wherein the polyether is poly(ethylene oxide) (PEO), poly(propylene oxide) (POP) or a copolymer (EO/PO), or wherein the polymer comprises crosslinked units derived from crosslinkable functional groups or crosslinked equivalents thereof, the crosslinkable functional group preferably being selected from acrylate, methacrylate, vinyl, glycidyl, and mercapto functional groups.

38-41. (canceled)

42. The solid electrolyte of claim 36, wherein the polymer is present at a concentration of from about 0.1 wt. % to about 20 wt. %, or from about 1 wt. % to about 15 wt. %, or from about 2 wt. % to about 10 wt. % in the solid electrolyte.

43. The solid electrolyte of claim 1, which further comprises an additive.

44. The solid electrolyte of claim 43, wherein the additive is a fluorinated compound comprising an amide function preferably being of formula R⁶X⁶C(O)N(H)X⁷R⁷, where R⁶ and R⁷ are independently alkyl, cycloalkyl, heterocycloalkyl, aryl, or heteroaryl groups, X⁶ is O, NH, or is absent, and X⁷ is absent or is a C(O), S(O)₂, or Si(R⁸R⁹) group, where R⁸ and R⁹ are alkyl groups, and where at least one of R⁶, R⁷, R⁸, and R⁹ is a group substituted by one or more fluorine atoms, and wherein R⁶ is a perfluorinated group and X⁶ is absent.

45-46. (canceled)

47. The solid electrolyte of claim 43, wherein the additive is present at a concentration of from about 5 wt. % to about 40 wt. %, or from about 10 wt. % to about 35 wt. %, or from about 15 wt. % to about 30 wt. % in the solid electrolyte.

48. An electrochemical cell comprising a negative electrode, a positive electrode, and an electrolyte, wherein the electrolyte is as defined in claim 1.

49. The electrochemical cell of claim 48, wherein the positive electrode comprises a positive electrode material comprising a positive electrode electrochemically active material, the positive electrode material preferably being on a current collector.

50. (canceled)

51. The electrochemical cell of claim 49, wherein the positive electrode electrochemically active material is:
(i) selected from metal phosphates, lithiated metal phosphates, metal oxides, and lithiated metal oxides; or
(ii) LiM′PO₄ where M′ is Fe Ni Mn Co or a combination of at least two thereof, LiV₃O₈, V₂O₅F, LiV₂O₅, LiMn₂O₄, LiM″O₂, where M″ is Mn, Co, Ni, or a combination of at least two thereof (such as NMC, LiMnₓCo_{y}Ni_{z}O₂ with x+y+z=1), Li(NiM′″)O₂ (where M″′ is Mn, Co, Al, Fe, Cr, Ti, Zr, or a combination of at least two thereof), sulfur, elemental selenium, elemental iodine, iron(III) fluoride, copper(II) fluoride, lithium iodide, carbon-based active materials such as graphite, organic cathode active materials, or a combination of at least two thereof, when compatible with each other.

52. (canceled)

53. The electrochemical cell of claim 49, wherein the positive electrode material further comprises an electronically conductive material, a binder, a salt, an ionic bifunctional molecule, and/or inorganic particles.

54. The electrochemical cell of claim 48, wherein the negative electrode comprises a negative electrode material comprising a negative electrode electrochemically active material, the negative electrode material preferably being on a current collector.

55. (canceled)

56. The electrochemical cell of claim 54, wherein the negative electrode electrochemically active material comprises a metal film comprising an alkali or alkaline earth metal or an alloy comprising an alkali or alkaline earth metal, preferably the alkali metal is selected from lithium and sodium.

57. (canceled)

58. The electrochemical cell of claim 54, wherein the negative electrode electrochemically active material comprises an intermetallic compound (for example, SnSb, TiSnSb, Cu₂Sb, AlSb, FeSb₂, FeSn₂, and CoSn₂), metal oxide, metal nitride, metal phosphide, metal phosphate (for example, LiTi₂(PO₄)₃), metal halide (for example, metal fluoride), metal sulfide, metal oxysulfide, carbon (for example, graphite, graphene, reduced graphene oxide, hard carbon, soft carbon, exfoliated graphite, and amorphous carbon), silicon (Si), a silicon-carbon composite (Si—C), a silicon oxide (SiOₓ), a silicon oxide-carbon composite (SiOₓ—C), tin (Sn), a tin-carbon composite (Sn—C), a tin oxide (SnOₓ), a tin oxide-carbon composite (SnOₓ—C), and combinations thereof, where compatible, and preferably wherein:
(i) the metal oxide is selected from compounds of formulae M″″_{b}O_{c}, (where M″″ is Ti, Mo, Mn, Ni, Co, Cu, V, Fe, Zn, Nb, or a combination thereof, and b and c are numbers such that the ratio c:b is in the range from 2 to 3) for example, MoO₃, MoO₂, MoS₂, V₂O₅, and TiNb₂O₇), spinel oxides (for example, NiCo₂O₄, ZnCo₂O₄, MnCo₂O₄, CuCo₂O₄, and CoFe₂O₄) and LiM′″″O (where M′″″ is Ti, Mo, Mn, Ni, Co, Cu, V, Fe, Zn, Nb, or a combination thereof) (for example, lithium titanate (such as Li₄Ti₅O₁₂) or lithium molybdenum oxide (such as Li₂Mo₄O₁₃)); or
(ii) the negative electrode material further comprises an electronically conductive material, a binder, a salt, an ionic bifunctional molecule, and/or inorganic particles.

59-60. (canceled)

61. A battery comprising at least one electrochemical cell as defined in claim 48, wherein said battery is preferably selected from the group consisting of a lithium battery, a lithium-ion battery, a sodium battery, a sodium-ion battery, a potassium battery, a potassium-ion battery, a magnesium battery, and a magnesium-ion battery, and more preferably a lithium battery or a lithium-ion battery.

62-64. (canceled)
